(19) United States
(12) Patent Application Publication
(10) Pub. No.: US 2015/0376702 A1
(43) Pub. Date: Dec. 31, 2015

(54) METHODS FOR ASSESSING THE RISK OF CANINE ATOPIC DERMATITIS

(71) Applicants: THE BROAD INSTITUTE, INC., Cambridge, MA (US); BIOCISTRONIX AB, Uppsala (SE)

(72) Inventors: Goran ANDERSSON, Uppsala (SE); Kerstin BERGVALL, Bromma (SE); Ake HEDHAMMER, Vaxholm (SE); Marcin KIERCZAK, Orbyhus (SE); Katarina TENGVALL, Upplands Vasby (SE); Kerstin LINDBLAD-TOH, Malden, MA (US)

(73) Assignees: BIOCISTRONIX AB, Uppsala (SE); THE BROAD INSTITUTE, INC., Cambridge, MA (US)

(21) Appl. No.: 14/768,538

(22) PCT Filed: Feb. 21, 2014
(86) PCT No.: PCT/US2014/017647

§ 371 Date: Aug. 18, 2015

Related U.S. Application Data
(60) Provisional application No. 61/767,990, filed on Feb. 22, 2013.

(51) Int. Cl. C12Q 1/68

(52) U.S. Cl. CPC C12Q 1/6883; C12Q 2600/156

(57) ABSTRACT

The present invention relates to methods for assessing the risk of a dog to develop canine atopic dermatitis. The methods comprise detecting in a sample of DNA obtained from a dog the presence or absence of at least one genetic marker, wherein said at least one genetic marker is located on dog (Canis familiaris) chromosome 27, said marker being associated with an increased risk of developing canine atopic dermatitis.

The present application claims the priority benefit of U.S. provisional application No. 61/767,990, filed Feb. 22, 2013, the entire contents of which are incorporated herein by reference.

### BACKGROUND OF THE INVENTION

I. Field of the Invention

The present invention relates to methods for assessing the risk of a dog to develop canine atopic dermatitis. The methods comprise detecting in a sample of DNA obtained from a dog the presence or absence of at least one genetic marker, wherein said at least one genetic marker is located on dog chromosome 27, said marker being associated with an increased risk of developing canine atopic dermatitis.

II. Background of the Invention

The domestic dog (Canis familiaris) has been bred for different purposes and characteristics for thousands of years. [1]. The creation of dog breeds started around 200 years ago and was based on few founders and breeding strategies such as strong selection for certain traits, popular sires and inbreeding/backcrossing. This has led to enrichment of disease mutations in different breeds. The German shepherd dog (GSD) breed has an exceptionally high susceptibility to immunological diseases or immune-related disorders including skin as well as gastrointestinal problems. Inflammatory and immune-related diseases that have been reported with high incidence in GSDs are, for example, exocrine pancreas insufficiency due to atrophy [2,3], canine atopic dermatitis (CAD) [4,5], anal furunculosis [6,7] and disseminated aspergillosis [8]. A predisposition for food hypersensitivity and bacterial folliculitis [9] as well as low serum IgA levels [10,11,12] have also been reported in the GSD breed. Several other dog breeds have increased risk for developing CAD and examples of such breeds are Golden Retriever, Labrador Retriever, West Highland white terrier, Boxer and Bullterrier [5, 14].

CAD is defined as an inflammatory and pruritic allergic skin disease caused by an interaction between genetic and environmental factors [13,14]. The characteristic clinical features are most commonly associated with IgE antibodies directed towards environmental allergens [15]. In dogs, the allergic symptoms appear as eczematous skin but do not show the sequential development called atopic march (eczema in a child being often followed by asthma and allergic rhinitis in the adult patient) as described in humans [16,17]. Clinical signs usually develop at a young age in both humans [16] and dogs. In dogs the onset is typically between six months and three years of age [18]. The initial signs of CAD can be seasonal or non-seasonal, depending on the allergens involved. Face, ears, paws, extremities, ventrum and flex-zones are typically affected by pruritus and erythema [18] in a pattern similar to that observed in human atopic dermatitis (AD) [19]. To establish the diagnosis of CAD an extensive work-up is required [20], where conditions with similar clinical presentations must be ruled out. These include: scabies or other pruritic ectoparasite infestations, pruritic bacterial skin infections, Malassezia dermatitis, flea allergy dermatitis and, less commonly, cornification disorders and contact dermatitis. Cutaneous adverse food reactions (CAFR) can present similarly or contribute to clinical signs of CAD, but can be mediated by either hypersensitivity or non-immunological reactions. Thus, ideally the presence of CAFR should be evaluated before making the diagnosis. Also scabies could satisfy many of the inclusion criteria [21] and therefore has to be excluded as possible differential diagnosis. A positive allergen-specific IgE test (serology or intradermal test) is needed for final diagnosis and aids in defining offending allergens. Different types of ichtyosis have been described in various breeds such as Golden retrievers [26], Cavalier King Charles spaniel [27] and Soft Coated Weaten terrier [28], however, to the inventors' knowledge, not in GSDs.

Immunoglobulin A (IgA) consists of two different forms, secretory IgA and serum IgA. In humans, serum concentrations of IgA are normally around 2-3 g/l, which makes it the second most prevalent antibody in serum after IgG [31]. IgA deficiency (IgAD) is the most common primary immunodeficiency in Caucasians with an estimated frequency of 1/600. IgA levels <0.07 g/l together with normal levels of IgG and IgM define IgAD in humans [32]. Compared to other dog breeds, very low IgA levels are known to be overrepresented in GSDs [33,34,35,36,37]. Low serum IgA levels have also been reported in Shar-Pei [38] and Beagle [39]. Moreover, low levels of secretory-IgA in mucosa, tears [11,40] and faecal extracts [41] have been reported in GSDs. Human studies show that children tend to have lower serum IgA levels than adults [42]. This is in concordance to the lower serum and secretory (tear) IgA levels being described in one year old or younger dogs compared to older dogs [43]. While increased incidence of upper respiratory tract infections, allergies and autoimmune diseases are observed in IgA-deficient human patients; more often humans show no symptoms at low levels of IgA [44]. Similarly, dogs with low IgA levels can either be asymptomatic or affected with recurrent upper respiratory infections and chronic dermatitis [39].

### SUMMARY OF THE INVENTION

The aim of the present investigation was to detect loci associated with CAD and evaluate whether IgA levels in serum are correlated with the CAD phenotype in GSDs. A strong correlation between serum IgA levels and CAD was found and a genome-wide significant association of a locus with CAD could be identified using serum IgA levels and age at sampling as covariates. Another aim of the investigation was to assess the frequency of the identified CAD risk locus in other dog breeds known to have increased risk for developing CAD, i.e., Golden Retriever, Labrador Retriever, West Highland white terrier, Boxer and Bullterrier.

Accordingly the present invention provides methods for assessing the risk of a dog to develop canine atopic dermatitis. The method can comprise obtaining a sample from said dog to be tested. The methods can comprise extracting DNA from a sample obtained from a dog.

The method can comprise determining in said DNA the allele of at least one genetic marker, wherein said at least one genetic marker is located in the region between the flanking SNPs at nucleotide positions 17,684,410 corresponding to position 201 in SEQ ID NO: 1 and position 19,292,898 corresponding to position 201 in SEQ ID NO:2 on dog (Canis familiaris) chromosome 27, CFA 27.

In particular, the genetic marker is selected from the SNPs listed in Tables 3 and 4. Most particularly, the genetic marker is selected from the SNPs listed in Table 3.

The method can comprise the step of identifying in said DNA the nucleotide in one or more specific position(s) selected from the positions (here given from the CanFam 2.0 assembly):
i) the nucleotide G and/or T in a nucleotide position corresponding to position 18,934,038 on CFA27, which corresponds to position 201 in SEQ ID NO: 3,
ii) the nucleotide C and/or A in a nucleotide position corresponding to position 18,934,219 on CFA27, which corresponds to position 201 in SEQ ID NO: 4,
iii) the nucleotide G and/or T in a nucleotide position corresponding to position 19,140,837 on CFA27, which corresponds to position 201 in SEQ ID NO: 5,
iv) the nucleotide G and/or T in a nucleotide position corresponding to position 19,142,893 on CFA27, which corresponds to position 201 in SEQ ID NO: 6,
v) the nucleotide A and/or T in a nucleotide position corresponding to position 19,121,205 on CFA27, which corresponds to position 201 in SEQ ID NO: 7,
vi) the nucleotide A and/or C in a nucleotide position corresponding to position 18,861,228 on CFA27, which corresponds to position 201 in SEQ ID NO: 8,
vii) the nucleotide C and/or A in a nucleotide position corresponding to position 18,964,049 on CFA27, which corresponds to position 201 in SEQ ID NO: 9,
viii) the nucleotide A and/or C in a nucleotide position corresponding to position 18,965,475 on CFA27, which corresponds to position 201 in SEQ ID NO: 10,
ix) the nucleotide A and/or G in a nucleotide position corresponding to position 18,486,594 on CFA27, which corresponds to position 201 in SEQ ID NO: 11,
x) the nucleotide T and/or C in a nucleotide position corresponding to position 19,29,2898 on CFA27, which corresponds to position 201 in SEQ ID NO: 2,
xi) the nucleotide T and/or C in a nucleotide position corresponding to position 19,048,938 on CFA27, which corresponds to position 201 in SEQ ID NO: 12,
xii) the nucleotide A and/or G in a nucleotide position corresponding to position 19,049,048 on CFA27, which corresponds to position 201 in SEQ ID NO: 13,
xiii) the nucleotide A and/or C in a nucleotide position corresponding to position 18,134,508 on CFA27, which corresponds to position 201 in SEQ ID NO: 14,
xiv) the nucleotide T and/or C in a nucleotide position corresponding to position 19,067,992 on CFA27, which corresponds to position 201 in SEQ ID NO: 15,
xv) the nucleotide A and/or G in a nucleotide position corresponding to position 18,161,172 on CFA27, which corresponds to position 201 in SEQ ID NO: 16,
xvi) the nucleotide G and/or A in a nucleotide position corresponding to position 18,699,406 on CFA27, which corresponds to position 201 in SEQ ID NO: 17,
xvii) the nucleotide A and/or C in a nucleotide position corresponding to position 18,874,358 on CFA27, which corresponds to position 201 in SEQ ID NO: 18,
xviii) the nucleotide T and/or A in a nucleotide position corresponding to position 19,264,902 on CFA27, which corresponds to position 201 in SEQ ID NO: 19,
xix) the nucleotide G and/or A in a nucleotide position corresponding to position 18,223,070 on CFA27, which corresponds to position 201 in SEQ ID NO: 20,
xx) the nucleotide G and/or A in a nucleotide position corresponding to position 18,804,142 on CFA27, which corresponds to position 201 in SEQ ID NO: 21,
xxi) the nucleotide A and/or C in a nucleotide position corresponding to position 18,582,103 on CFA27, which corresponds to position 201 in SEQ ID NO: 22,
xxii) the nucleotide T and/or C in a nucleotide position corresponding to position 18,131,103 on CFA27, which corresponds to position 201 in SEQ ID NO: 23,
xxiii) the nucleotide A and/or T in a nucleotide position corresponding to position 18,207,512 on CFA27, which corresponds to position 201 in SEQ ID NO: 24,
xxiv) the nucleotide C and/or T in a nucleotide position corresponding to position 18,581,634 on CFA27, which corresponds to position 201 in SEQ ID NO: 25,
xxv) the nucleotide T and/or C in a nucleotide position corresponding to position 17,944,696 on CFA27, which corresponds to position 201 in SEQ ID NO: 26,
xxvi) the nucleotide A and/or G in a nucleotide position corresponding to position 18,082,732 on CFA27, which corresponds to position 201 in SEQ ID NO: 27,
xxvii) the nucleotide T and/or G in a nucleotide position corresponding to position 18,443,579 on CFA27, which corresponds to position 201 in SEQ ID NO: 28,
xxviii) the nucleotide A and/or C in a nucleotide position corresponding to position 17,751,542 on CFA27, which corresponds to position 201 in SEQ ID NO: 29,
xxix) the nucleotide A and/or T in a nucleotide position corresponding to position 17,760,444 on CFA27, which corresponds to position 201 in SEQ ID NO: 30,
xxx) the nucleotide C and/or T in a nucleotide position corresponding to position 18,581,490 on CFA27, which corresponds to position 201 in SEQ ID NO: 31,
xxxi) the nucleotide C and/or G in a nucleotide position corresponding to position 17,848,875 on CFA27, which corresponds to position 201 in SEQ ID NO: 32,
xxxii) the nucleotide A and/or G in a nucleotide position corresponding to position 18,207,618 on CFA27, which corresponds to position 201 in SEQ ID NO: 33,
xxxiii) the nucleotide G and/or A in a nucleotide position corresponding to position 19,097,445 on CFA27, which corresponds to position 201 in SEQ ID NO: 34,
xxxiv) the nucleotide T and/or C in a nucleotide position corresponding to position 19,118,236 on CFA27, which corresponds to position 201 in SEQ ID NO: 35,
xxxv) the nucleotide A and/or G in a nucleotide position corresponding to position 17,716,804 on CFA27, which corresponds to position 201 in SEQ ID NO: 36,
xxxvi) the nucleotide G and/or A in a nucleotide position corresponding to position 19,007,501 on CFA27, which corresponds to position 201 in SEQ ID NO: 37,
xxxvii) the nucleotide C and/or T in a nucleotide position corresponding to position 19,021,017 on CFA27, which corresponds to position 201 in SEQ ID NO: 38,
xxxviii) the nucleotide A and/or C in a nucleotide position corresponding to position 19,048,269 on CFA27, which corresponds to position 201 in SEQ ID NO: 39,
xxxix) the nucleotide A and/or G in a nucleotide position corresponding to position 17,684,210 on CFA27, which corresponds to position 201 in SEQ ID NO: 1, and
xl) the nucleotide G and/or A in a nucleotide position corresponding to position 18,605,999 on CFA27, which corresponds to position 201 in SEQ ID NO: 40,
   wherein the presence of said first nucleotide in said position indicates an increased risk for said dog of developing CAD.

In accordance, the presence of said second nucleotide in said position indicates a decreased risk for said dog of developing CAD.

The term “effective,” as that term is used in the specification and/or claims (e.g., “an effective amount,” means adequate to accomplish a desired, expected, or intended result.

“Treatment” and “treating” as used herein refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of a condition. This includes, but is not limited to, a reduction in the frequency or severity of the signs or symptoms of a disease.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIE DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Manhattan plot from the association analysis of CAD with IgA levels and age at sampling as covariates shows a significant association on dog chromosome 27.

FIGS. 2A-B. (FIG. 2A) Dog chromosome 27 is displayed with association score for each SNP and minor allele frequencies (MAF) below. (FIG. 2B) The SNPs in high LD (r²≧0.8) with the top SNP are marked and the whole associated region is indicated by the outer dotted lines with the genes displayed below. The two top SNPs (shaded area) surround the PKP2 gene.

FIGS. 3A-B. Fine mapping of the dog chromosome 27 locus confirms the association with CAD and further pinpoints the region around the PKP2 gene. The association (p-value after 1,000,000 permutations) of the genotyped SNPs (n=42) and haplotypes (n=11) are presented in panel A and B, respectively.

### DESCRIPTION OF ILLUSTRATIVE EMBODIMENTS

The dog to be tested in the methods according to the present invention can be selected from any dog or breed of dogs belonging to the species Canis familiaris, such as Affenpinscher, Afghan Hound, Airedale Terrier, Akita, Alaskan Malamute, American Cocker Spaniel, American Eskimo Dog, American Foxhound, American Staffordshire Terrier, American Water Spaniel, Anatolian Shepherd, Australian Cattle Dog, Australian Shepherd, Australian Terrier, Basenji, Basset Hound, Beagle, Bearded Collie, Beauceron, Bedlington Terrier, Belgian Malinois, Belgian Sheepdog, Belgian Tervuren, Bernese Mountain Dog, Bichon Frise, Black Russian Terrier, Black and Tan Coonhound, Bloodhound, Border Collie, Border Terrier, Borzoi, Boston Terrier, Bouvier des Flandres, Boxer, Briard, Brittany, Brussels Griffon, Bull Terrier, Bulldog, Bullmastiff, Cairn Terrier, Canaan Dog, Cardigan Welsh Corgi, Cavalier King Charles Spaniel, Chesapeake Bay Retriever, Chihuahua, Chinese Crested Dog, Chinese Shar-Pei, Chow Chow, Clumber Spaniel, Collie, Curly-Coated Retriever, Dachshund, Dalmatian, Dandie Dinmont Terrier, Doberman Pinscher, English Cocker Spaniel, English Foxhound, English Setter, English Springer Spaniel, English Toy Spaniel, Field Spaniel, Finnish Spitz, Flat-Coated Retriever, French Bulldog, German Pinscher, German Shepherd Dog, German Shorthaired Pointer, German Wirehaired Pointer, Giant Schnauzer, Glen of Imaal Terrier, Golden Retriever, Gordon Setter, Great Dane, Great Pyrenees, Greater Swiss Mountain Dog, Greyhound, Harrier, Havanese, Ibizan Hound, Irish Setter, Irish Terrier, Irish Water Spaniel, Irish Wolfhound, Italian Greyhound, Japanese Chin, Keeshond, Kerry Blue Terrier, Komondor, Kuvasz, Labrador Retriever, Lakeland Terrier, Lhasa Apso, Lowchen, Maltese, Manchester Terrier, Mastiff, Miniature Bull Terrier, Miniature Pinscher, Miniature Schnauzer, Neapolitan Mastiff, Newfoundland, Norfolk Terrier, Norwegian Elkhound, Norwich Terrier, Nova Scotia Duck Tolling Retriever, Old English Sheepdog, Otterhound, Papillon, Parson Russell Terrier, Pekingese, Pembroke Welsh Corgi, Petit Basset Griffon Vendeen, Pharaoh Hound, Plott, Pointer, Polish Lowland Sheepdog, Pomeranian, Poodle, Portuguese Water Dog, Pug, Puli, Redbone Coonhound, Rhodesian Ridgeback, Rottweiler, Saint Bernard, Samoyed, Schipperke, Scottish Deerhound, Scottish Terrier, Sealyham Terrier, Shetland Sheepdog, Shiba Inu, Shih Tzu, Siberian Husky, Silky Terrier, Skye Terrier, Smooth Fox Terrier, Soft Coated Wheaten Terrier, Spinone Italiano, Staffordshire Bull Terrier, Standard Schnauzer, Sussex Spaniel, Tibetan Mastiff, Tibetan Spaniel, Tibetan Terrier, Toy Fox Terrier, Vizsla, Weimaraner, Welsh Springer Spaniel, Welsh Terrier, West Highland White Terrier, Whippet, Wire Fox Terrier, Wirehaired Pointing Griffon, Yorkshire Terrier. In particular, the dog is a German Shepherd dog or any other dog affected by CAD or a dog from a breed with increased risk of developing CAD. In particular, the dog is a German Shepherd Dog.

The term “sample” or “biological sample” according to the present invention refers to any material containing nucleated cells from said dog to be tested. In a particular embodiment the biological sample to be used in the methods of the present invention is selected from the group consisting of blood, sperm, hair roots, milk, body fluids as well as tissues including nucleated cells.

DNA extraction, isolation and purification methods are well-known in the art and can be applied in the present invention. Standard protocols for the isolation of genomic DNA are inter alia referred to in Sambrook, J., Russell. D. W. Molecular Cloning: A Laboratory Manual, the third edition, Cold Spring Harbor Laboratory Press, Cold Spring Harbor. New York, 1.31-1.38, 2001 and Sharma. R. C., et al. “A rapid procedure for isolation of RNA-free genomic DNA from mammalian cells”, BioTechniques, 14. 176-178. 1993.

According to the present invention the term “SNP” refers to a single nucleotide polymorphism at a particular position in the dog genome that varies among a population of individuals. SNPs can be identified by their location within the disclosed particular sequence, i.e., within the interval of Ser. No. 17/684,410 (corresponding to position 201 in SEQ ID NO: 1) and 19,292,898 (corresponding to position 201 in SEQ ID NO: 2) on dog chromosome 27 (CFA 27). SNPs identified as being useful for assessing the risk for a dog to develop canine atopic dermatitis according to the present invention are shown in Tables 3, 4, and 5. For example, the first SNP listed in Table 3 indicates that the nucleotide base (or the allele) at nucleotide position 18,934,038 on dog chromosome 27 of the reference sequence as referred to herein may be either Guanosine (G) or Thymidine (T). The allele associated with or indicative for an increased risk of the dog to develop canine atopic dermatitis is in this case Guanosine (G).

The term “detecting in said DNA the presence or absence of at least one genetic marker” in accordance with the present invention refers to a method for determining or identifying whether a particular nucleotide sequence is present in a DNA sample. There are several methods known by those skilled in the art for determining whether such nucleotide sequence is present in a DNA sample. These include the amplification of a DNA segment encompassing the genetic marker by means of the polymerase chain reaction (PCR) or any other amplification method, interrogate the genetic marker by means of allele-specific hybridization, the 3′exonuclease assay (Taqman assay), fluorescent dye and quenching agent-based PCR assay, the use of allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader (Third wave technologies), ligase chain reaction assays, 5′-nuclease assay-based techniques, hybridization capillary array electrophoresis (CAE), protein truncation assays (PTT), immunoassays, and solid phase hybridization (dot blot, reverse dot blot, chips). This list of methods is not meant to be exclusive, but just to illustrate the diversity of available methods. Some of these methods can be performed in accordance with the methods of the present invention in microarray format (microchips) or on beads.

The invention thus also relates to isolated nucleic acid probes, primers or primer pairs and their use in the methods according to the invention, wherein the probes, primers or primer pairs hybridize(s) under stringent conditions to the DNA comprising the interval between 17,684,410 (corresponding to position 201 in SEQ ID NO: 1) and 19,292,898 (corresponding to position 201 in SEQ ID NO: 2) on dog chromosome 27 (CFA 27), or to the complementary strand thereof.

In particular, the probes, primers or primer pairs hybridize(s) under stringent conditions to any one of the sequences SEQ ID NO: 1 to 40, or to the complementary strand thereof.

In particular, the primers of the invention have a length of at least 14 nucleotides such as 17 or 21 nucleotides.

In one embodiment, the probes or primers actually binds to the position of the SNPs as referred to in Tables 3 and 4, i.e. to position 201 of any one of the sequences SEQ ID NO: 1 to 40. Such an allele specific oligonucleotide in accordance with the present invention is typically an oligonucleotide of at least 14 to 21 nucleotide bases in length designed to detect a difference of a single base in the target's genetic sequence of the dog to be tested. In accordance with the present invention one or more specific primers can be applied in order to identify more than a single SNP as referred to herein. As a consequence, when binding is performed under stringent conditions, such primer or such primers is/are useful to distinguish between different polymorphic variants as binding only occurs if the sequences of the primer and the target have full complementarily. In particular, the primers have a maximum length of 24 nucleotides. Such primers can be coupled with an appropriate detection method such as an elongation reaction or an amplification reaction, which may be used to differentiate between the polymorphic variants and then used to assess the risk of the dog to develop canine atopic dermatitis.

Hybridization may be performed under stringent or highly stringent conditions. “Stringent or highly stringent conditions” of hybridization are well known to or can be established by the person skilled in the art according to conventional protocols. Appropriate stringent conditions for each sequence may be established on the basis of well-known parameters such as temperature, composition of the nucleic acid molecules, salt conditions, etc.; see, for example, Sambrook et al., “Molecular Cloning, A Laboratory Manual”, CSH Press, Cold Spring Harbor, 1989 or Higgins and Hames (eds.), “Nucleic acid hybridization, a practical approach”, IRL Press, Oxford 1985, see in particular the chapter “Hybridization Strategy” by Britten & Davidson. Typical (highly stringent) conditions comprise hybridization at 65° C. in 0.5×SSC and 0.1% SDS or hybridization at 42° C. in 50% formamide, 4×SSC and 0.1% SDS. Hybridization is usually followed by washing to remove unspecific signals. Washing conditions include conditions such as 65° C., 0.2×SSC and 0.1% SDS or 2×SSC and 0.1% SDS or 0.3×SSC and 0.1% SDS at 25° C.-65° C.

The term “nucleotide positions 17,684,410 and 19,292,898 base pairs on dog chromosome 27” and other similar denoted nucleotide positions refer to the dog reference sequence according to the draft assembly CanFam2.0. [45].

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

### Sampling and Ethics Statement

Blood samples (EDTA for DNA extraction and serum for IgA measurements) were collected from 207 German shepherd pet dogs in collaboration with veterinary clinics throughout Sweden. Owner consent was collected for each dog. Sampling was conformed to the approval of the Swedish Animal Ethical Committee (no. C62/10) and the Swedish Animal Welfare Agency (no. 31-1711/10).

### Samples

Genomic DNA was extracted from the EDTA blood samples using the Qiagen mini- and/or midiprep extraction kit (Qiagen, Hilden, Germany). DNA samples were diluted in de-ionized water and stored at −20° C. Serum was separated from the red blood cells by centrifugation and then stored at −20/−80° C.

### CAD Phenotype Characterisation

The CAD cases were dogs of all ages with positive reactions on allergen-specific IgE test (intradermal test or IgE serology test), either with or without concurrent cutaneous adverse food reactions (CAFR). Clinical diagnoses were established by first ruling out other causes of pruritus such as ectoparasite infestation, staphylococcal pyoderma and Malassezia dermatitis. A hypoallergenic dietary trial (at least 6-8 weeks followed by a challenge period) was then conducted in order to evaluate the potential contribution of CAFR. Atopic reactions were concluded if the dog was not adequately controlled on hypoallergenic diet and had positive reactions on intradermal allergy tests (skin prick test) or IgE serology tests.

All CAD controls were over five years of age and never suffered from pruritus, repeated ear inflammations or skin lesions compatible with CAD, neither prior to nor at the time of sampling. The age cut-off for CAD controls was set at five since affected dogs rarely debut at ages older than 3 years of age [17,18]. The information was based on either owner questionnaire and/or clinical examination. In addition, the inventors excluded dogs with low IgA levels (IgA≦0.10 g/l) as CAD controls.

### Measurements of Serum IgA

Serum IgA concentrations were measured with enzyme-linked immunosorbent assay (ELISA) using polyclonal goat anti-dog IgA antibodies (AbD Serotec), polyclonal mouse anti-dog IgA antibodies (AbD Serotec) and polyclonal, AP-conjugated goat anti-mouse IgG (Jackson Immunoresearch). All antibodies were diluted 1:2,000 in PBS and the serum samples were diluted 1:25.000; 1:50.000 and 1:100,000 in PBS. All samples were measured at least two times. The coefficient of variation (CV) was calculated. Samples with a CV value ≧15% were measured again. Before the average concentration was calculated, potentially outlying concentrations were excluded. With a maximal variation of 15% the reproducibility of the inventors' measurements are in the lower range of ELISA measurements which can be as high as 25%.

Dogs with serum IgA levels ≦0.10 g/l were considered to be IgA deficient and thus not deemed appropriate controls for CAD. All the dogs were sampled at the age of more than one year except for one individual that was 11 months and 13 days at the time of sampling.

### Statistical Analyses of Traits and Covariates

The relationships between measured phenotypes and other possible covariates were examined and Fisher's exact test used for count data to determine whether CAD-gender relationships were significant. Similarly the Welch two-sample t-test was used for determining the CAD-IgA levels relationship. The same approaches were used to check if there were any significant differences in CAD status or IgA levels between subpopulations.

As IgA levels may vary with age, the inventors fitted a linear model to determine the age effect on the IgA levels, and used Pearson's correlation coefficient to measure the strength of the relationship. CAD cases and controls were considered separately and together. The age at the time of sampling was defined at 0.1-year resolution for most individuals and estimated at a year resolution for 10 dogs (n_{controls}=7, n_{cases}=3).

### SNP Genotyping and Quality Control

The initial data set consisted of 207 individuals genotyped using the Illumina 170K CanineHD BeadChip (Illumina, USA). Summary of individuals in each trait class is presented in Table 1, before and after quality control (QC). Prior to principal genome-wide association studies (GWAS), iterative QC was performed to remove poorly genotyped and noisy data. Out of the initial number of 174,376 SNP markers, 55,399 (31.77%) non-informative markers (minor allele frequency (MAF) below 1%), 2,537 (1.45%) were excluded due to call rate below 0.95 and 2,722 (1.56%) markers due to the departure from Hardy-Weinberg equilibrium (first p<1×10⁻⁸ and then FDR <0.2 in CAD controls only). In total, 114,348 markers (65.57%) were included in both analyses.

Considering the whole dataset consisting of 207 GSD individuals, two individuals were excluded due to exceptionally high identity-by-state, IBS >0.95 (the one with lowest call rate was excluded in each pair—all were CAD cases) and two apparent outliers on the multidimensional scaling (MDS) plot resulting in 203 individuals passing QC. After QC, 25 individuals in total were excluded from the association analysis; five were missing CAD status, five CAD controls had low IgA levels and 15 CAD controls were missing IgA levels (Table 1).

The initial association (with IgA level and age at sampling as covariates) indicated population stratification (λ=1.3, λse=1.5×10⁻³). Hence, it was decided to perform a closer examination of the genetic structure of the inventors' GSD population by computing autosomal genomic kinship matrix and performing standard K-means clustering. In order to determine the number of clusters (subpopulations), the inventors performed a number of K-means clustering with K={1, 2, . . . , 10}. At each iteraction, the sum of within-cluster sums of squares (ΣWCSS) was computed and stored. Subsequently, the so-called screen test by plotting ΣWCSS vs. K was used and the number of clusters (K=2) corresponding to the first inflection point chosen (for details see: [56]). The clusters define subpopulations.

### Genome-Wide Association Analysis

Association analysis of CAD (91 cases and 88 controls) with IgA levels and age at sampling as covariates were performed. The GenABEL package ver. 1.7-0 [57], a part of R statistical suite/software, ver. 2.14.2 [58] were used for the genome-wide association analyses. The mixed model approach for all the final analysis presented was used. Mixed models were fitted using polygenic_hglm function from the hglm package ver. 1.2-2 [59]. All parameters used for functional calls are discussed in the paragraphs describing particular steps of the previous sections. p-values below 0.05 (p_{raw}) were considered as significant and after 100,000 permutations as genome-wide significant p-values (p_{genome}).

For haplotype definitions LD-clumping (settings; r²=0.8, p1=0.0001, p2=0.001, distance d=3 Mb) the inventors performed using the inventors' own R implementation of the algorithm described in the PLINK documentation (PLINK v1.07, [60]) and Haploview 4.2 (version 1.0).

### Targeted Re-Sequencing

Targeted capture of in total 6.5 Mb out of which 2.8 Mb spanning chromosome 27:16.8-19.6 Mb (CanFam 2.0) including the ˜1.5 Mb associated haplotype, was performed using a 385K custom-designed sequence capture array from Roche NimbleGen. Hybridization library preparation was performed as described by Olsson et al. [61]. Captured enriched libraries were sequenced with a read length of 100 bp (paired-end reads), using HiSeq 2000 (Illumina sequencing technology). Sequencing was performed by the SNP&SEQ Technology Platform at SciLifeLab Uppsala. Obtained reads were mapped to CanFam 2.0 [45] using Burrows-Wheeler Aligner [62]. The Genome Analysis Toolkit (GATK) (the world-wide-web at broadinstitute.org/gatk) was used for base quality recalibration and local realignment and the tool picard (hosted by SAMtools [63]) for removing PCR duplicates. For variant calling SAMtools/0.1.18 was applied using mpileup format and bcftools. Maximum read depth to call a SNP (−D) was set to 300 and the function −C50 was applied to reduce the effect of reads with excessive mismatches (http://samtools.sourceforge.net). Mean coverage in the seven analyzed individuals was 61.4 reads and mean share of positions covered by at least 10 reads was 88%.

SEQScoring [64] (the world-wide-web at seqscoring.net) was used to score the SNPs by conservation and haplotype pattern; and the integrative genomics viewer (IGV) [65] was used for manual visualization of SNPs, individual coverage and indels. In total, 8,765 SNPs were identified in the chromosome 27 region. Out of these, 2,587 SNPs followed the pattern of the case and control haplotypes defined by the top GWAS SNPs. The pattern was based on three dogs homozygous for the control haplotype, one dog homozygous for the case haplotype and three dogs carrying the case and control haplotype (i.e., carriers of the case haplotype). Out of the 2,587 SNPs only 46 SNPs were located within conserved elements (+/−5 bp) as scored by SEQscoring according to SiPhy constraint elements detected by the alignment of 29 eutherian mammals [66]. The inventors picked out 60 SNPs for designing a genotyping array. The selection was based on the following criteria; 40 SNPs out of the 46 SNPs stated above (SNPs too close to each other and located in repeated sequences were excluded), SNPs from the genome-wide array for comparison, manually picked SNPs within the PKP2 gene (not conserved) and SNPs in gaps in order to cover the entire associated region. Out of these, 54 SNPs were successfully pooled for additional genotyping in all dogs.

### Genotyping of Fine Mapping SNPs

The 54 SNPs were genotyped using iPLEX Sequenom MassARRAY platform (on the world-wide-web at sequenom.com/iplex) in 185 GSD dogs After analyzing the quality of the SNP genotyping 12 SNPs were excluded due to bad calling; nine due to heterozygotes were incorrectly called as homozygous and two due to one of the homozygous genotypes was falsely called as heterozygotes and one due to MAF=0. In total 42 SNPs were left for the analysis. For the association analysis of the genotyped SNPs and for defining haplotypes the inventors used Haploview 4.2 (version 1.0). In total, 84 controls and 91 cases were included in the analysis—the same set as in the genome-wide association analysis of CAD except for 4 excluded controls (2 were not included due to missing DNA and 2 were excluded due to low call rate=48%). The genotyping of Golden retriever, Labrador retriever, West Highland white terrier, Boxer and Bullterrier was performed exactly as for German Shepherds as described above.

### Example 2

### Results

### Characterization of the Sample Cohort

The diagnostic features CAD and low IgA levels were investigated in a Swedish population of GSDs. The total number of dogs included in the study is presented in Table 1.

TABLE 1
Individuals classified with CAD in the
final analysis (before QC in brackets)

| | CAD | CAD | CAD | |
|---|---|---|---|---|
| IgA levels | cases | controls | missing | |
| ≧0.20 g/l | 21 (22) | 57 (57) | 0 (3) | |
| 0.10-0.20 g/l | 33 (35) | 31 (31) | 0 (0) | |
| IgA ≦0.10 g/l | 37 (37) | 0 (5) | 0 (1) | |
| IgA missing | 0 (0) | 0 (15) | 0 (1) | |
| Total | 91 (94) | 88 (108) | 0 (5) | 179 (207) |

When considering the CAD phenotype the relationship of the following parameters; CAD status, IgA levels and gender. 40.7% (n=37) of the CAD cases had IgA-levels ≦0.10 g/l compared to 5.4% (n=5) of the CAD controls was first evaluated. The IgA levels were significantly lower in CAD cases versus controls p=1.1×10⁻⁵, mean IgA level in cases was 0.16 g/l and 0.26 g/l in controls (before excluding the 5 CAD controls with low IgA levels from the final association analysis, see Materials and Methods). No gender bias in cases versus controls for CAD (p=0.88) was detected. When considering whether IgA levels were related to age, regression coefficient of 0.42 in all dogs together (p=3.0×10⁻⁹), 0.37 in cases (p=3.6×10⁴) and 0.28 in controls (p=8.5×10⁻³) were determined. The age at sampling was added as a covariate in the association analyses in order to remove any confounding effects of the IgA measurements' dependency of age.

### Genome-Wide Association Studies (GWAS)

Genotyping of ˜170,000 SNP markers of the entire GSD cohort (n=207) was performed. Non-informative markers and markers with low call rate were excluded, giving 114,348 markers for the final analysis. An association analysis of CAD using IgA levels and age at sampling as covariates was performed.

A Locus on Chromosome 27 Associated with CAD

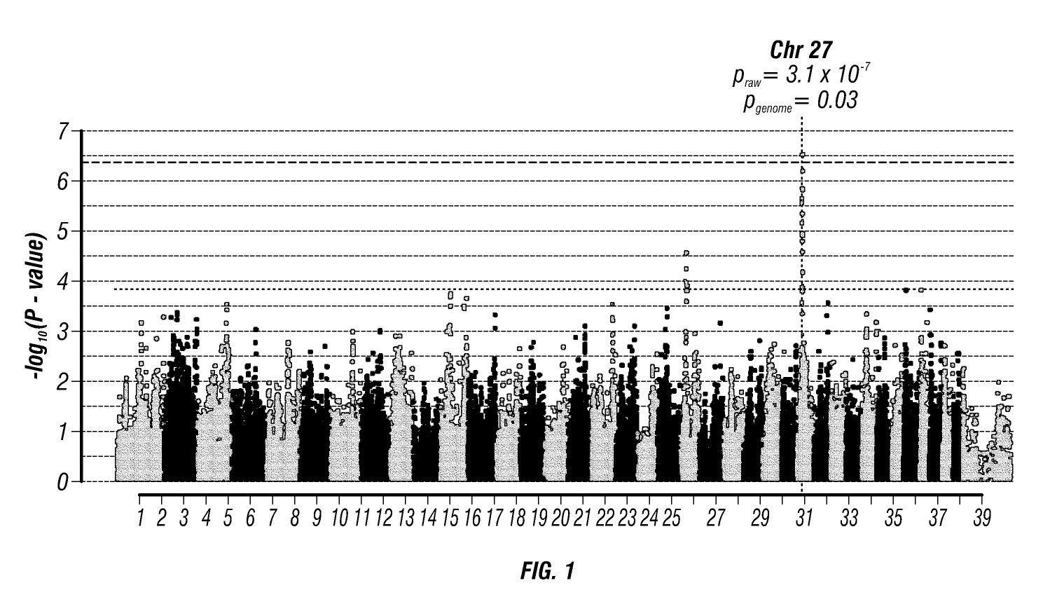

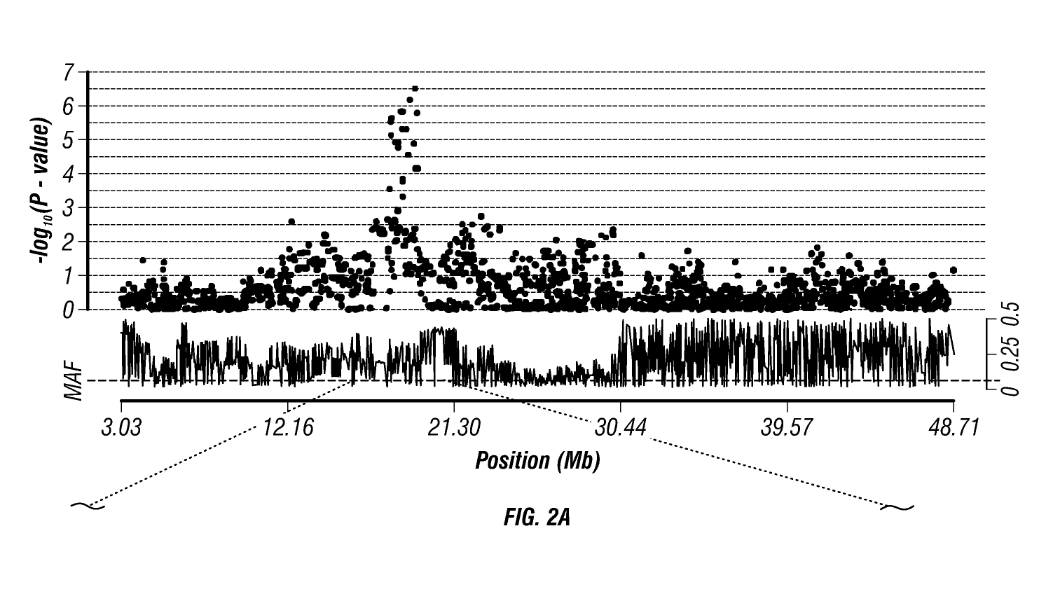

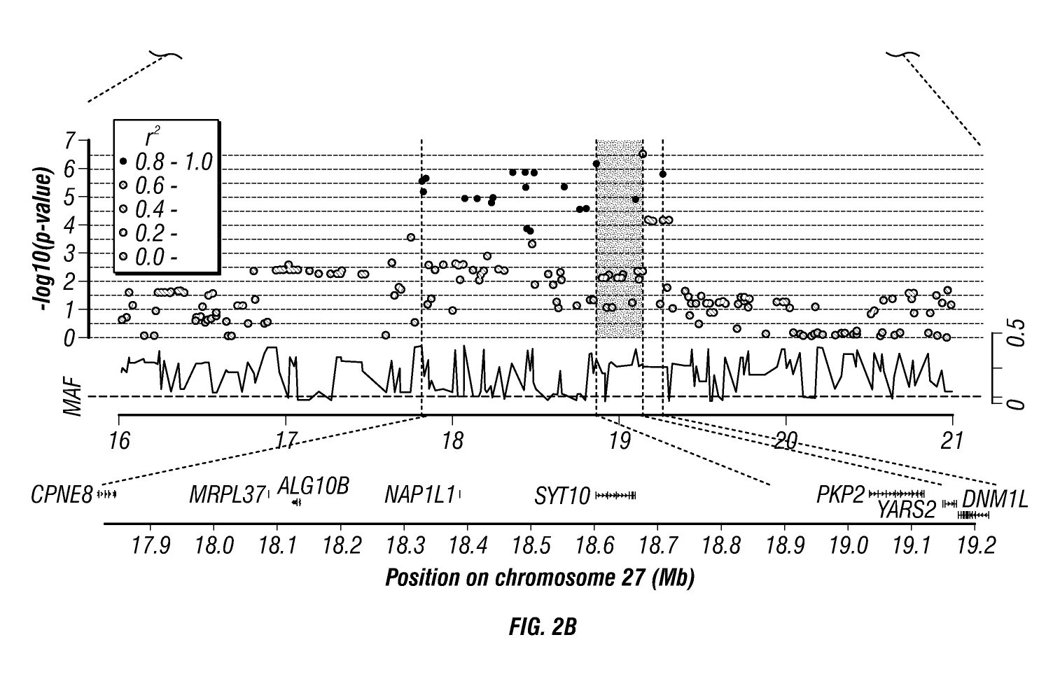

In the association analysis of CAD the inventors found a significant association to chromosome 27 where 19 SNPs between 17,814,493 and 19,262,027 (CanFam 2.0) showed association p<2.8×10⁻⁵. The top two SNPs are located at Chr 27: 19,140,837 bp (p^{raw}=3.1×10⁻⁷ and p^{genome}=0.03) and 18,861,228 bp (p^{raw}=6.7×10⁻⁷ and p^{genome}=0.07) (FIGS. 1 and 2A-B). To define the associated haplotype clumping using r²=0.8 was performed, and a 21 SNP haplotype spanning from Ser. No. 17/814,493 to Ser. No. 19/262,027 identified. This haplotype region contains 9 genes (CPNE8, MRPC37, ALG10B, ALG10, NAP1L1, SYT10, PKP2, YARS2 and DNM1L) where the two top SNPs surround the PKP2 gene as indicated in FIG. 2B. The haplotype corresponds to the region identified by the 19 associated SNPs and covers a region of ˜1.5 Mb. The haplotype region shows a mosaic pattern of association very typical for purebred dogs [45], thus it is not possible from this data to define a shorter associated haplotype.

Using Haploview lower association to CAD was detected when using the ˜1.5 Mb haplotype compared to the single top SNPs (p^{haplotype}=2.6×10⁻⁵). The observed MAF of the top SNP (Chr 27: 19,140,837 bp) was 0.29 across all samples, and 0.40 and 0.16 in cases and controls, respectively. The minor allele (G) conferred an OR=1.28 for CAD.

### Targeted Re-Sequencing of the Associated Locus on Chromosome 27

A targeted re-sequencing of the locus on chromosome 27 spanning 16.8-19.6 Mb (CanFam 2.0) i.e. including the associated haplotype located at ˜17.8-19.3 Mb was performed. In total, three dogs homozygous for the control haplotype, one dog homozygous for the case haplotype and three dogs heterozygous for the case and control haplotypes were sequenced. In total, 2,587 SNPs of all the identified SNPs (n=8,765) followed the case and control haplotype pattern (see Example 1). As expected, the majority of the SNPs detected to correlate with the case/control haplotypes (86%) were located within the associated (17.8-19.3 Mb) region. In total, 54 SNPs were included on an iPLEX array for further genotyping in the same cohort used for the genome-wide association study. These SNPs were concordant with the risk haplotype and considered functional candidates based on their location in conserved elements or in genes. In addition the top GWAS SNPs were included. For the final analysis, 42 SNPs and 84 controls and 91 cases remained after quality control (see Example 1). Using Haploview, haplotypes based on r²≧0.9 between neighbouring SNPs the inventors defined. The risk alleles of block 11 and 7 (GCCA and AGG, respectively) had a frequency of 40.1% in the cases versus 16.7% in the controls (p^{raw}=1.3×10⁻⁶, p^{1,000,000perm}=4.0×10⁻⁶). The common control allele TTT of block 11 had the same p-value as the risk allele and a frequency of 83.3% in controls versus 59.9% in cases. Considering single SNPs; the top associated were the risk alleles of Ser. No. 18/934,038 by and 18,934,219 bp (part of block 7), and 19,140,837 bp (part of block 11 and also the top GWAS SNP). They had the same frequency as the risk alleles of the corresponding haplotypes and were associated to the same extent (p^{raw}=1.3×10⁻⁶) but with a slightly less significant p-value after permutations (p^{1,000,000perm}=1.4×10⁻⁵) due to the larger number of SNPs compared to haplotypes. See the association analysis results of haplotypes and SNPs in Tables 2 and 3, respectively.

TABLE 2
Top 10 haplotype alleles from the association
analysis of fine-mapping data

| | | Frequency | | | |
|---|---|---|---|---|---|
| Block | Allele | Case | Control | p-value | p_{1,000,000 permutations} |
| 7 | GCCA | 0.401 | 0.167 | 1.3 × 10⁻⁶ | 4.0 × 10⁻⁶ |
| 11 | AGG | 0.401 | 0.167 | 1.3 × 10⁻⁶ | 4.0 × 10⁻⁶ |
| 11 | TTT | 0.599 | 0.833 | 1.3 × 10⁻⁶ | 4.0 × 10⁻⁶ |
| 7 | TAAC | 0.599 | 0.821 | 5.0 × 10⁻⁶ | 2.8 × 10⁻⁵ |
| 9 | TAT | 0.418 | 0.208 | 2.7 × 10⁻⁵ | 0.0001 |
| 4 | AA | 0.378 | 0.179 | 3.7 × 10⁻⁵ | 0.0002 |
| 9 | CGC | 0.582 | 0.786 | 4.6 × 10⁻⁵ | 0.0003 |
| 4 | CG | 0.622 | 0.810 | 1.0 × 10⁻⁴ | 0.0009 |
| 6 | TTC | 0.824 | 0.940 | 8.0 × 10⁻⁴ | 0.006 |
| 3 | AT | 0.170 | 0.060 | 0.0013 | 0.0079 |

TABLE 3
Top 15 SNP alleles from the association analysis of fine-mapping data

| Position on | | | | | | |
|---|---|---|---|---|---|---|
| CanFam2.0 | Risk | Frequency | | | p_{1,000,000} | Ref |
| CFA 27 | allele | Case | Control | p-value | ₚₑᵣₘᵤₜₐₜᵢₒₙₛ | allele |
| 18934038⁷ | G | 0.401 | 0.167 | 1.3 × 10⁻⁶ | 1.4 × 10⁻⁵ | T |
| 18934219⁷ | C | 0.401 | 0.167 | 1.3 × 10⁻⁶ | 1.4 × 10⁻⁵ | A |
| 19140837¹¹ | G | 0.401 | 0.167 | 1.3 × 10⁻⁶ | 1.4 × 10⁻⁵ | T |
| 19142893¹¹ | G | 0.400 | 0.167 | 1.5 × 10⁻⁶ | 1.9 × 10⁻⁵ | T |
| 19121205¹¹ | A | 0.401 | 0.169 | 1.8 × 10⁻⁶ | 2.5 × 10⁻⁵ | T |
| 18861228 | A | 0.390 | 0.167 | 3.5 × 10⁻⁶ | 4.6 × 10⁻⁵ | C |
| 18964049⁷ | C | 0.401 | 0.179 | 5.0 × 10⁻⁶ | 6.1 × 10⁻⁵ | A |
| 18965475⁷ | A | 0.401 | 0.179 | 5.0 × 10⁻⁶ | 6.1 × 10⁻⁵ | C |
| 18486594 | A | 0.390 | 0.173 | 6.8 × 10⁻⁶ | 7.6 × 10⁻⁵ | G |
| 19292898 | T | 0.401 | 0.185 | 9.4 × 10⁻⁶ | 0.00010 | C |
| 19048938 | T | 0.417 | 0.208 | 3.0 × 10⁻⁵ | 0.00030 | C |
| 19049048 | A | 0.417 | 0.208 | 3.0 × 10⁻⁵ | 0.00030 | G |
| 18134508 | A | 0.378 | 0.179 | 3.7 × 10⁻⁵ | 0.00040 | C |
| 19067992 | T | 0.418 | 0.214 | 4.6 × 10⁻⁵ | 0.00050 | C |
| 18161172 | A | 0.378 | 0.190 | 0.00010 | 0.0014 | G |

⁷SNPs part of block 7,
¹¹SNPs part of block 11

TABLE 4
SNP alleles

| Position | Risk allele | p-value | Ref allele |
|---|---|---|---|
| 18699406 | G | 0.00010 | A |
| 18874358 | A | 0.00010 | C |
| 19264902 | T | 0.00020 | A |
| 18223070 | G | 0.00040 | A |
| 18804142 | G | 0.0010 | A |
| 18582103 | A | 0.0011 | C |
| 18131103 | T | 0.0013 | C |
| 18207512 | A | 0.0013 | T |
| 18581634 | C | 0.0013 | T |
| 17944696 | T | 0.0020 | C |
| 18082732 | A | 0.0023 | G |
| 18443579 | T | 0.0023 | G |
| 17751542 | A | 0.0025 | C |
| 17760444 | A | 0.0025 | T |
| 18581490 | C | 0.0026 | T |
| 17848875 | C | 0.0034 | G |
| 18207618 | A | 0.0036 | G |
| 19097445 | G | 0.0057 | A |
| 19118236 | T | 0.0057 | C |
| 17716804 | A | 0.0072 | G |
| 19007501 | G | 0.0104 | A |
| 19021017 | C | 0.0104 | T |
| 19048269 | A | 0.0154 | C |
| 17684210 | A | 0.0184 | G |
| 18605999 | G | 0.019 | A |

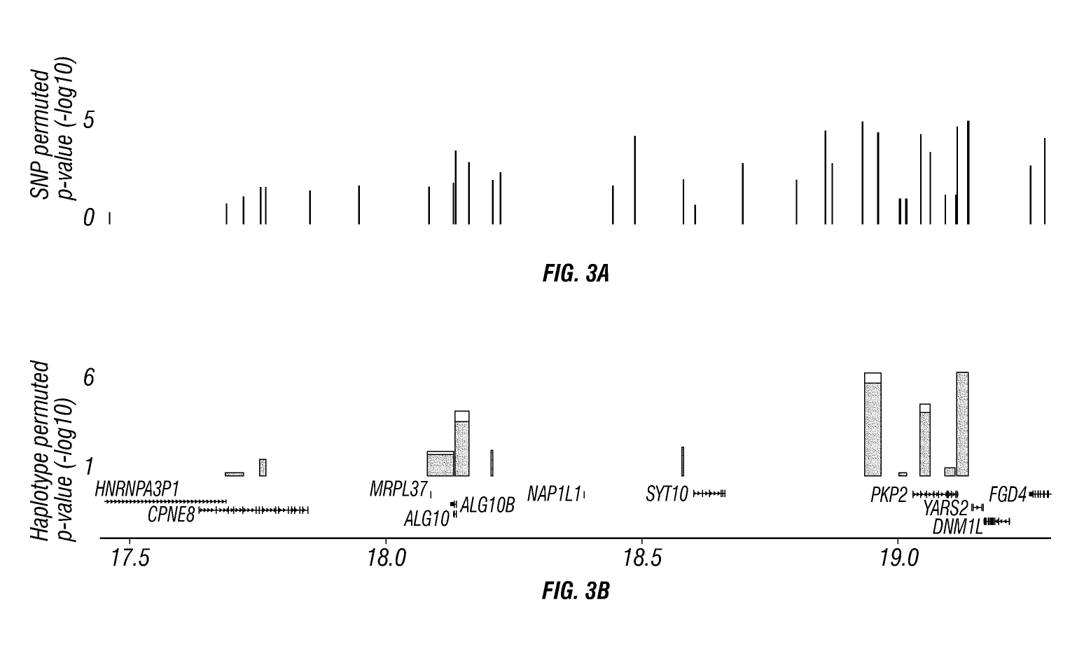

The association of SNPs and haplotypes (p-value after 1,000,000 permutations) as well as the defined haplotypes and the LD plot are visualized in FIGS. 3A-B. These results indicate that the region 18,934,038-19,142,893 Mb harbours the causative mutation predisposing for CAD in the studied GSD population. This is in concordance with the genome-wide association results where the top associated SNP is located at 19,140,837 bp. Only one gene, PKP2, falls within the top region (defined by block 7-11). The PKP2 gene, encoding the protein Plakophilin 2, a central component of desmosomes [46], is an excellent candidate gene for CAD.

### Analysis of the Top-Associated CAD SNPs in Other High-Risk Dog Breeds

Two of the SNPs identified in the German Shepherd (SNPs at position 18964049 and 18965475) were genotyped in dogs of five additional breeds selected on the basis of having intermediate to high risk for developing CAD additional breeds. The results are presented in Table 5.

TABLE 5
Genetic analysis of German Shepherd-associated
SNPs for PKP2 in other breeds

| | | | | | |
|---|---|---|---|---|---|
| | cases | cases | controls | controls | |
| Breed | (n) | (freq) | (n) | (freq) | conclusion |

SNP position: CFA27: 18964049 - risk allele C

| | | | | | |
|---|---|---|---|---|---|
| Golden | 10 | 0.90 | 15 | 0.61 | Risk allele |
| retrievers | | | | | associated |
| | | | | | (P = 0.024) |
| Labrador | 54 | 1.00 | 100 | 1.00 | Risk allele fixed |
| retrievers | | | | | |
| West | 66 | 1.00 | 32 | 1.00 | Risk allele fixed |
| highland | | | | | |
| white terriers | | | | | |
| Boxer | 54 | 0.97 | 24 | 1.00 | Risk allele |
| | | | | | almost fixed |
| Bullterriers | 20 | 1.00 | 7 | 1.00 | Risk allele fixed |

SNP position: CFA27: 18965475 - risk allele A

| | | | | | |
|---|---|---|---|---|---|
| Golden | 10 | 0.95 | 15 | 0.68 | Risk allele |
| retrievers | | | | | associated |
| | | | | | (P = 0.022) |
| Labrador | 54 | 1.00 | 100 | 1.00 | Risk allele fixed |
| retrievers | | | | | |
| West | 66 | 1.00 | 32 | 1.00 | Risk allele fixed |
| highland | | | | | |
| white terriers | | | | | |
| Boxer | 54 | 0.97 | 24 | 1.00 | Risk allele |
| | | | | | almost fixed |
| Bullterriers | 20 | 1.00 | 7 | 1.00 | Risk allele fixed |

Haplotype frequency 18964049-C/18965475-A

| | | | | | |
|---|---|---|---|---|---|
| Golden | 10 | 0.90/0.95 | 15 | 0.61/0.68 | P = 0.02 |
| retrievers | | | | | |
| Labrador | 54 | 1.00 | 100 | 1.00 | fixed |
| retrievers | | | | | |
| West | 66 | 1.00 | 32 | 1.00 | fixed |
| highland | | | | | |
| white terriers | | | | | |
| Boxer | 54 | 0.97 | 24 | 1.00 | almost fixed |
| Bullterriers | 20 | 1.00 | 7 | 1.00 | fixed |

It was found that the risk allele of both these two SNPs were the dominating alleles in both cases and in controls in four of these breeds, where the risk allele seems to be fixed. In Golden retrievers there was a slight association between the risk alleles of these two SNPs and CAD.

### Example 3

### Discussion

### Genome-Wide Association of CAD

The inventors detected a significant difference in IgA levels in CAD cases compared to CAD controls. This suggests a functional role of IgA in the aetiology of CAD. The overall low IgA levels seen in the GSD breed might contribute to its predisposition for CAD: among the CAD cases 40.7% had low IgA-levels compared to only 5.4% of the CAD controls. The associated haplo-type on chromosome 27 from the genome-wide association analysis of CAD includes nine genes; CPNE8, MRPC37, ALG10B, ALG10, NAP1L1, SYT10, PKP2, YARS2 and DNM1L.

### Candidate Mutation Detection and Validation Genotyping of the CFA27 Associated Region

The nucleotide sequencing data generated in the 2.8 Mb region on chromosome 27 verified the ˜1.5 Mb long associated haplotype showing 86% of the 2,587 SNPs following the case and control haplotype pattern located at −17.8-19.3 Mb. Based on further genotyping of 42 SNPs within the region there is clear indication that the region 18.94-19.14 Mb, based on both haplotypes and single SNPs, harbours the mutation predisposing for CAD in GSDs. By performing targeted re-sequencing of the associated region the inventors attempted to identify all variants concordant with the phenotype and then evaluate their potential as the risk variant. Here, the inventors identified two haplotypes with multiple SNPs with equally strong association and a potential for function. While one or several of these variants may be the causative variant, it is also possible that the actual mutation may have been missed in the targeted sequencing process or in the genotyping process as several SNPs failed genotyping for technical reasons.

Furthermore, the top-associated SNPs in the PKP2 locus were genotyped in cases and healthy control dogs from five other dog breeds known to be at increased risk for developing CAD (Table 5). In Golden retriever, a significant genetic association was shown (p=0.02). In Labrador retriever, West Highland white terrier, Boxer and Bullterrier the risk alleles were fixed in the study population whereas in Boxer the risk alleles were almost fixed. The allele frequencies of these SNPs remain to be defined in different subpopulations of these breeds as well as in other dog breeds with different prevalence of CAD development. CAD development, like other complex diseases is dependent on multiple genetic risk factors and environmental risk factors. Importantly, the possibility to test for the PKP2 risk genotype allows assessment of interaction between additional segregating genetic risk factors.

Furthermore, the ability to predict functionality is not comprehensive as functional variants may be located in non-conserved elements or in complicated regions with low sequence coverage. The actual functional variant may also be an indel or CNV not picked up in this analysis. Further analysis will reveal the exact causative mutation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Karlsson E K, Baranowska I, Wade C M, Salmon Hillbertz N H, Zody M C, et al. (2007) Efficient mapping of mendelian traits in dogs through genome-wide association. Nature genetics 39: 1321-1328.
2. Westermarck E, Saari S A, Wiberg M E (2010) Heritability of exocrine pancreatic insufficiency in German Shepherd dogs. Journal of veterinary internal medicine/American College of Veterinary Internal Medicine 24: 450-452.
3. Moeller E M, Steiner J M, Clark L A, Murphy K E, Famula T R, et al. (2002) Inheritance of pancreatic acinar atrophy in German Shepherd Dogs. American journal of veterinary research 63: 1429-1434.
4. Jaeger K, Linek M, Power H T, Bettenay S V, Zabel S, et al. (2010) Breed and site predispositions of dogs with atopic dermatitis: a comparison of five locations in three continents. Veterinary dermatology 21: 118-122.
5. Nodtvedt A, Egenvall A, Bergvall K, Hedhammar A (2006) Incidence of and risk factors for atopic dermatitis in a Swedish population of insured dogs. The Veterinary record 159: 241-246.
6. Day M J W B (1992) Pathology of surgically resected tissue from 305 cases of anal furunculosis in the dog. Journal of Small Animal Practice 33: 583-589.
7. Day M J (1993) Immunopathology of anal furunculosis in the dog. Journal of Small Animal Practice 34: 381-389.
8. Day M J, Penhale W J, Eger C E, Shaw S E, Kabay M J, et al. (1986) Disseminated aspergillosis in dogs. Australian veterinary journal 63: 55-59.
9. Scott D W, Paradis M (1990) A survey of canine and feline skin disorders seen in a university practice: Small Animal Clinic, University of Montreal, Saint-Hyacinthe, Quebec (1987-1988). The Canadian veterinary journal La revue veterinaire canadienne 31: 830-835.
10. Whitbread T J, Batt R M, Garthwaite G (1984) Relative deficiency of serum IgA in the german shepherd dog: a breed abnormality. Research in veterinary science 37: 350-352.
11. Batt R M, Barnes A, Rutgers H C, Carter S D (1991) Relative IgA deficiency and small intestinal bacterial overgrowth in German shepherd dogs. Research in veterinary science 50: 106-111.
12. Willard M D, Simpson R B, Fossum T W, Cohen N D, Delles E K, et al. (1994) Characterization of naturally developing small intestinal bacterial overgrowth in 16 German shepherd dogs. Journal of the American Veterinary Medical Association 204: 1201-1206.
13. Meury S, Molitor V, Doherr M G, Roosje P, Leeb T, et al. (2011) Role of the environment in the development of canine atopic dermatitis in Labrador and golden retrievers. Vet Dermatol 22: 327-334.
14. Nodtvedt A, Guitian J, Egenvall A, Emanuelson U, Pfeiffer D U (2007) The spatial distribution of atopic dermatitis cases in a population of insured Swedish dogs. Prey Vet Med 78: 210-222.
15. Olivry T, DeBoer D J, Griffin C E, Halliwell R E, Hill P B, et al. (2001) The ACVD task force on canine atopic dermatitis: forewords and lexicon. Veterinary immunology and immunopathology 81: 143-146.
16. Zheng T, Yu J, Oh M H, Zhu Z (2011) The atopic march: progression from atopic dermatitis to allergic rhinitis and asthma. Allergy, asthma & immunology research 3: 67-73.
17. Favrot C, Steffan J, Seewald W, Picco F (2010) A prospective study on the clinical features of chronic canine atopic dermatitis and its diagnosis. Vet Dermatol 21: 23-31.
18. Griffin C E, DeBoer D J (2001) The ACVD task force on canine atopic dermatitis (XIV): clinical manifestations of canine atopic dermatitis. Veterinary Immunology and Immunopathology 81: 255-269.
19. Brown S, Reynolds N J (2006) Atopic and non-atopic eczema. BMJ 332: 584-588.
20. Olivry T (2010) New diagnostic criteria for canine atopic dermatitis. Vet Dermatol 21: 123-126.
21. DeBoer D J, Hillier A (2001) The ACVD task force on canine atopic dermatitis (XV): fundamental concepts in clinical diagnosis. Veterinary Immunology and Immunopathology 81: 271-276.
22. Irvine A D, McLean W H, Leung D Y (2011) Filaggrin mutations associated with skin and allergic diseases. N Engl J Med 365: 1315-1327.
23. Boguniewicz M, Leung D Y M (2010) Recent insights into atopic dermatitis and implications for management of infectious complications. Journal of Allergy and Clinical Immunology 125: 4-13.
24. Palmer C N, Ismail T, Lee S P, Terron-Kwiatkowski A, Zhao Y, et al. (2007) Filaggrin null mutations are associated with increased asthma severity in children and young adults. J Allergy Clin Immunol 120: 64-68.
25. Smith F J, Irvine A D, Terron-Kwiatkowski A, Sandilands A, Campbell L E, et al. (2006) Loss-of-function mutations in the gene encoding filaggrin cause ichthyosis vulgaris. Nat Genet 38: 337-342.
26. Grall A, Guaguere E, Planchais S, Grond S, Bourrat E, et al. (2012) PNPLA1 mutations cause autosomal recessive congenital ichthyosis in golden retriever dogs and humans. Nat Genet 44: 140-147.
27. Alheidari Z, Ortonne J P, A. P (1994) Congenital ichthyosis in two cavalier King Charles spaniel littermates. Vet Dermatol 5: 117-121.
28. Helman R G, Rames D S, Chester D K (1996) Ichthyosiform dermatosis in a soft-coated wheaten terrier. Vet Dermatol 8: 53-58.
29. Betz R C, Pforr J, Flaquer A, Redler S, Hanneken S, et al. (2007) Loss-of-function mutations in the filaggrin gene and alopecia areata: strong risk factor for a severe course of disease in patients comorbid for atopic disease. Journal of Investigative Dermatology 127: 2539-2543.
30. Tobin D J, Gardner S H, Luther P B, Dunston S M, Lindsey N J, et al. (2003) A natural canine homologue of alopecia areata in humans. Br J Dermatol 149: 938-950.
31. Woof J M, Kerr M A (2006) The function of immunoglobulin A in immunity. The Journal of pathology 208: 270-282.
32. Hammarstrom L S, C.I.E. (Oxford 2007) Genetic approach to common variable immunodeficiency and IgA deficiency. Oxford University Press In Ochs, H. D., Smith, C. I. E. and Puck, J. M. (Eds.): 1207-1216.
33. Whitbread T J, Batt R M, Garthwaite G (1984) Relative deficiency of serum IgA in the german shepherd dog: a breed abnormality. Res Vet Sci 37: 350-352.
34. Batt R M, Barnes A, Rutgers H C, Carter S D (1991) Relative IgA deficiency and small intestinal bacterial overgrowth in German shepherd dogs. Res Vet Sci 50: 106-111.
35. German A J, Hall E J, Day M J (1998) Measurement of IgG, IgM and IgA concentrations in canine serum, saliva, tears and bile. Vet Immunol Immunopathol 64: 107-121.
36. Peters I R, Calvert E L, Hall E J, Day M J (2004) Measurement of immunoglobulin concentrations in the feces of healthy dogs. Clin Diagn Lab Immunol 11: 841-848.
37. Littler R M, Batt R M, Lloyd D H (2006) Total and relative deficiency of gut mucosal IgA in German shepherd dogs demonstrated by faecal analysis. Vet Rec 158: 334-341.
38. Moroff S D, Hurvitz A I, Peterson M E, Saunders L, Noone K E (1986) IgA deficiency in shar-pei dogs. Veterinary immunology and immunopathology 13: 181-188.
39. Felsburg P J, Glickman L T, Jezyk P F (1985) Selective IgA deficiency in the dog. Clinical immunology and immunopathology 36: 297-305.
40. Day M J (1996) Low IgA concentration in the tears of German shepherd dogs. Australian Veterinary Journal 74: 433-434.
41. Littler R M, Batt R M, Lloyd D H (2006) Total and relative deficiency of gut mucosal IgA in German shepherd dogs demonstrated by faecal analysis. The Veterinary record 158: 334-341.
42. Janzi M, Kull I, Sjoberg R, Wan J, Melen E, et al. (2009) Selective IgA deficiency in early life: association to infections and allergic diseases during childhood. Clinical immunology 133: 78-85.
43. German A J, Hall E J, Day M J (1998) Measurement of IgG, IgM and IgA concentrations in canine serum, saliva, tears and bile. Veterinary immunology and immunopathology 64: 107-121.
44. Cunningham-Rundles C (2001) Physiology of IgA and IgA deficiency. Journal of clinical immunology 21: 303-309.
45. Lindblad-Toh K, Wade C M, Mikkelsen T S, Karlsson E K, Jaffe D B, et al. (2005) Genome sequence, comparative analysis and haplotype structure of the domestic dog. Nature 438: 803-819.
46. Mertens C, Kuhn C, Franke W W (1996) Plakophilins 2a and 2b: constitutive proteins of dual location in the karyoplasm and the desmosomal plaque. J Cell Biol 135: 1009-1025.
47. Willis M B (1980) German shepherd dog his survey. The Veterinary record 107: 565.
48. Bonne S, van Hengel J, van Roy F (2000) Assignment of the plakophilin-2 gene (PKP2) and a plakophilin-2 pseudogene (PKP2P1) to human chromosome bands 12p11 and 12p13, respectively, by in situ hybridization. Cytogenetics and cell genetics 88: 286-287.
49. Godsel L M, Dubash A D, Bass-Zubek A E, Amargo E V, Klessner J L, et al. (2010) Plakophilin 2 couples actomyosin remodeling to desmosomal plaque assembly via RhoA. Mol Biol Cell 21: 2844-2859.
50. Ishida-Yamamoto A, Igawa S, Kishibe M (2011) Order and disorder in corneocyte adhesion. J Dermatol 38: 645-654.
51. Wood S H, Clements D N, Ollier W E, Nuttall T, McEwan N A, et al. (2009) Gene expression in canine atopic dermatitis and correlation with clinical severity scores. Journal of dermatological science 55: 27-33.
52. Proksch E, Folster-Holst R, Jensen J M (2006) Skin barrier function, epidermal proliferation and differentiation in eczema. J Dermatol Sci 43: 159-169.
53. Marsella R, Olivry T, Carlotti D N (2011) Current evidence of skin barrier dysfunction in human and canine atopic dermatitis. Veterinary dermatology.
54. Marsella R, Samuelson D (2009) Unravelling the skin barrier: a new paradigm for atopic dermatitis and house dust mites. Veterinary dermatology 20: 533-540.
55. Rawlings A V, Scott I R, Harding C R, Bowser P A (1994) Stratum corneum moisturization at the molecular level. Journal of Investigative Dermatology 103: 731-741.
56. Everitt B S, Hothorn T (2010) A Handbook of Statistical Analyses Using R: Chapman & Hall/CRC.
57. Aulchenko Y S, Ripke S, Isaacs A, van Duijn C M (2007) GenABEL: an R library for genome-wide association analysis. Bioinformatics 23: 1294-1296.
58. Ihaka R (1998) A Brief History. R: Past and Future History. Statistics Department, The University of Auckland, Auckland, New Zealand: http://cran.r-projectorg/doc/html/interface98-paper/paper_{—}2. html. Accessed 20 Jun. 2012.
59. Rönnegård L, Shen X, Alam M (2010) hglm: A package for fitting hierchical generalized linear models. The R Journal 2: 20-28.
60. Purcell S, Neale B, Todd-Brown K, Thomas L, Ferreira M A, et al. (2007) PLINK: a tool set for whole-genome association and population-based linkage analyses. Am J Hum Genet 81: 559-575.
61. Olsson M, Meadows J R, Truve K, Rosengren Pielberg G, Puppo F, et al. (2011) A novel unstable duplication upstream of HAS2 predisposes to a breed-defining skin phenotype and a periodic fever syndrome in Chinese Shar-Pei dogs. PLoS Genet 7: e1001332.
62. Li H, Durbin R (2009) Fast and accurate short read alignment with Burrows-Wheeler transform. Bioinformatics 25: 1754-1760.
63. Li H, Handsaker B, Wysoker A, Fennell T, Ruan J, et al. (2009) The Sequence Alignment/Map format and SAMtools. Bioinformatics 25: 2078-2079.
64. Truvé K, Eriksson O, Norling M, Wilbe M, Mauceli E, et al. (2011) SEQscoring: a tool to facilitate the interpretation of data generated with next generation sequencing technologies. EMBnet journal 17.1: 38-45.
65. Robinson J T, Thorvaldsdottir H, Winckler W, Guttman M, Lander E S, et al. (2011) Integrative genomics viewer. Nat Biotechnol 29: 24-26.
66. Lindblad-Toh K, Garber M, Zuk O, Lin M F, Parker B J, et al. (2011) A high-resolution map of human evolutionary constraint using 29 mammals. Nature 478: 476-482.

What is claimed is:

1. A method for assessing the risk of a dog to develop canine atopic dermatitis, said method comprising the steps of:
i) extracting DNA from a sample obtained from said dog to be tested, and
ii) determining in said DNA an allele of at least one genetic marker, wherein said at least one genetic marker is located in the region between the flanking SNPs at nucleotide position 17,684,410 corresponding to position 201 in SEQ ID NO: 1 and nucleotide position 19,292,898 corresponding to position 201 in SEQ ID NO: 2 on dog (Canis familiaris) chromosome CFA 27.

2. The method according to claim 1, wherein the genetic marker is selected from the SNPs listed in Table 3 and Table 4.

3. The method according to claim 2, wherein the genetic marker is selected from the SNPs listed in Table 3.

4. The method according to claim 1, comprising detecting in said DNA the presence or absence of:
i) the nucleotide G and/or T in a nucleotide position corresponding to position 18,934,038 on CFA27, which corresponds to position 201 in SEQ ID NO: 3,
ii) the nucleotide C and/or A in a nucleotide position corresponding to position 18,934,219 on CFA27, which corresponds to position 201 in SEQ ID NO: 4,
iii) the nucleotide G and/or T in a nucleotide position corresponding to position 19,140,837 on CFA27, which corresponds to position 201 in SEQ ID NO: 5,
iv) the nucleotide G and/or T in a nucleotide position corresponding to position 19,142,893 on CFA27, which corresponds to position 201 in SEQ ID NO: 6,
v) the nucleotide A and/or T in a nucleotide position corresponding to position 19,121,205 on CFA27, which corresponds to position 201 in SEQ ID NO: 7,
vi) the nucleotide A and/or C in a nucleotide position corresponding to position 18,861,228 on CFA27, which corresponds to position 201 in SEQ ID NO: 8,
vii) the nucleotide C and/or A in a nucleotide position corresponding to position 18,964,049 on CFA27, which corresponds to position 201 in SEQ ID NO: 9,
viii) the nucleotide A and/or C in a nucleotide position corresponding to position 18,965,475 on CFA27, which corresponds to position 201 in SEQ ID NO: 10,
ix) the nucleotide A and/or G in a nucleotide position corresponding to position 18,486,594 on CFA27, which corresponds to position 201 in SEQ ID NO: 11:
x) the nucleotide T and/or C in a nucleotide position corresponding to position 19,29,2898 on CFA27, which corresponds to position 201 in SEQ ID NO: 2,
xi) the nucleotide T and/or C in a nucleotide position corresponding to position 19,048,938 on CFA27, which corresponds to position 201 in SEQ ID NO: 12,
xii) the nucleotide A and/or G in a nucleotide position corresponding to position 19,049,048 on CFA27, which corresponds to position 201 in SEQ ID NO: 13,
xiii) the nucleotide A and/or C in a nucleotide position corresponding to position 18,134,508 on CFA27, which corresponds to position 201 in SEQ ID NO: 14,
xiv) the nucleotide T and/or C in a nucleotide position corresponding to position 19,067,992 on CFA27, which corresponds to position 201 in SEQ ID NO: 15,
xv) the nucleotide A and/or G in a nucleotide position corresponding to position 18,161,172 on CFA27, which corresponds to position 201 in SEQ ID NO: 16,
xvi) the nucleotide G and/or A in a nucleotide position corresponding to position 18,699,406 on CFA27, which corresponds to position 201 in SEQ ID NO: 17,
xvii) the nucleotide A and/or C in a nucleotide position corresponding to position 18,874,358 on CFA27, which corresponds to position 201 in SEQ ID NO: 18,
xviii) the nucleotide T and/or A in a nucleotide position corresponding to position 19,264,902 on CFA27, which corresponds to position 201 in SEQ ID NO: 19,
xix) the nucleotide G and/or A in a nucleotide position corresponding to position 18,223,070 on CFA27, which corresponds to position 201 in SEQ ID NO: 20,
xx) the nucleotide G and/or A in a nucleotide position corresponding to position 18,804,142 on CFA27, which corresponds to position 201 in SEQ ID NO: 21,
xxi) the nucleotide A and/or C in a nucleotide position corresponding to position 18,582,103 on CFA27, which corresponds to position 201 in SEQ ID NO: 22,
xxii) the nucleotide T and/or C in a nucleotide position corresponding to position 18,131,103 on CFA27, which corresponds to position 201 in SEQ ID NO: 23,
xxiii) the nucleotide A and/or T in a nucleotide position corresponding to position 18,207,512 on CFA27, which corresponds to position 201 in SEQ ID NO: 24,
xxiv) the nucleotide C and/or T in a nucleotide position corresponding to position 18,581,634 on CFA27, which corresponds to position 201 in SEQ ID NO: 25,
xxv) the nucleotide T and/or C in a nucleotide position corresponding to position 17,944,696 on CFA27, which corresponds to position 201 in SEQ ID NO: 26,
xxvi) the nucleotide A and/or G in a nucleotide position corresponding to position 18,082,732 on CFA27, which corresponds to position 201 in SEQ ID NO: 27,
xxvii) the nucleotide T and/or G in a nucleotide position corresponding to position 18,443,579 on CFA27, which corresponds to position 201 in SEQ ID NO: 28,
xxviii) the nucleotide A and/or C in a nucleotide position corresponding to position 17,751,542 on CFA27, which corresponds to position 201 in SEQ ID NO: 29,
xxix) the nucleotide A and/or T in a nucleotide position corresponding to position 17,760,444 on CFA27, which corresponds to position 201 in SEQ ID NO: 30,
xxx) the nucleotide C and/or T in a nucleotide position corresponding to position 18,581,490 on CFA27, which corresponds to position 201 in SEQ ID NO: 31,
xxxi) the nucleotide C and/or G in a nucleotide position corresponding to position 17,848,875 on CFA27, which corresponds to position 201 in SEQ ID NO: 32,
xxxii) the nucleotide A and/or G in a nucleotide position corresponding to position 18,207,618 on CFA27, which corresponds to position 201 in SEQ ID NO: 33,
xxxiii) the nucleotide G and/or A in a nucleotide position corresponding to position 19,097,445 on CFA27, which corresponds to position 201 in SEQ ID NO: 34,
xxxiv) the nucleotide T and/or C in a nucleotide position corresponding to position 19,118,236 on CFA27, which corresponds to position 201 in SEQ ID NO: 35,
xxxv) the nucleotide A and/or G in a nucleotide position corresponding to position 17,716,804 on CFA27, which corresponds to position 201 in SEQ ID NO: 36,
xxxvi) the nucleotide G and/or A in a nucleotide position corresponding to position 19,007,501 on CFA27, which corresponds to position 201 in SEQ ID NO: 37,
xxxvii) the nucleotide C and/or T in a nucleotide position corresponding to position 19,021,017 on CFA27, which corresponds to position 201 in SEQ ID NO: 38,
xxxviii) the nucleotide A and/or C in a nucleotide position corresponding to position 19,048,269 on CFA27, which corresponds to position 201 in SEQ ID NO: 39,
xxxix) the nucleotide A and/or G in a nucleotide position corresponding to position 17,684,210 on CFA27, which corresponds to position 201 in SEQ ID NO: 1, and
xl) the nucleotide G and/or A in a nucleotide position corresponding to position 18,605,999 on CFA27, which corresponds to position 201 in SEQ ID NO: 40;
wherein the presence of said first nucleotide in said position indicates an increased risk for said dog of developing CAD.

5. An isolated nucleic acid probe, primer or a primer pair hybridizing to any one of the sequences SEQ ID NO: 1 to 40, or to the complementary strand thereof.

6. The isolated nucleic acid probe, primer or a primer pair according to claim 5, wherein hybridizing is performed under stringent conditions.


SEQUENCE LISTING (40 sequences)

SEQ ID NO: 1 - DNA, 401 bp, Canis familiaris
  misc_feature (201)..(201): n is a or g
aactaaacctttctgaacatttttatcaaaaacagaattcatctggttggtgccataaatagatgaatgttctactagcactgctcctccaaagcagtttaacaattagttgatttagaaatcaggaatatttccattctatcattttctgcaggacttgttcctaaattggaaaagtagacatttatccgattagctcangcataaaatagaggaaaactttcaaagacttgattgtgaattttattttcttttggccaaagttttaaaagaaacagtgttttgtgttccaataaaagtaatgtttacatacttttttcaaatgactgaaaactttcatacttgttgagtaaaaaaaaacttgcatacatttttcctcacatttttctaattatgggata

SEQ ID NO: 2 - DNA, 401 bp, Canis familiaris
  misc_feature (201)..(201): n is t or c
aatttccagtgaggttattttttggcagcaatattttactaaattggctgtgagacacatcttgctctttaaatcacaaggctgcctggccactggttaccccataatagaaagggagtgggagagaaacatttacttttagcatcattccagtccggagaatcagggggcaatttccttaggtagtccttcaggagcatntcataccggggaatccgctgaacaggttctagcatgtgatgctgcaaagtcaagctcccacagatcttctgtttctataatgagaaaggttttaaagagagataaaatgaagttggccaatctaaaaactcactgttgtacagtatttttctgtattttcagtattatcaaggacagtgtttccttacaaagggtggtgt

SEQ ID NO: 3 - DNA, 401 bp, Canis familiaris
  misc_feature (201)..(201): n is g or t
ctcagcctgaaaacaggattccctgatctgttttaagtatggcaatgttgattatagtttttactacccagatatctgcctccagctatttcatagtccccaggaggggtggagatagctgtacactggccatagttgtgacaaaaaggcatttagtgacaatctgcatcttgagaattttaaccctaattataggagtanaacccctaaaggaggcaaaaattaaatcccagaatgagtgtattggctcccagttgtgccagtaaaaactgattggtataaccatactaagcctggtactaccttcaggtgaaactgagaaaaatctcattcacctcagagggataagaaagggatattggactgtgtatgggaagggaccctaggaagctaaaatgtat

SEQ ID NO: 4 - DNA, 401 bp, Canis familiaris
  misc_feature (201)..(201): n is c or a
aaccctaattataggagtagaacccctaaaggaggcaaaaattaaatcccagaatgagtgtattggctcccagttgtgccagtaaaaactgattggtataaccatactaagcctggtactaccttcaggtgaaactgagaaaaatctcattcacctcagagggataagaaagggatattggactgtgtatgggaagggacnctaggaagctaaaatgtatgctgtttacttctctatggagcactacagagatttttgctaagggaaaaatgtaatgaggtaggcaattgtatgcaacccccaaaagagggtttgggggacctgattcaaaaaaggacagcaaatgataaaccatgattgccagggagctatcagatggaaaagaaaaattgtcaggagca

SEQ ID NO: 5 - DNA, 401 bp, Canis familiaris
  misc_feature (201)..(201): n is g or t
gttcaagatgagaaagctgaaacaatatggcaaaggaacttgctcaatgtgacagccagaaagaatcaaactgtgattcaagactatatttgtcttactccaaagcctgtatttttactgcactctgtggctcccctgactcagagagactataatcaggaccctcagaaggctagtgaagctgctagaccaggacaaagnctgcgggggttagcatgctccaactctcagagagaggaagcacatggttatcatgacttgcaacagcaaagcagatattaagatgcaactccttgccagaggctctctgcaaaattcaaactgtgggtgagcagatcaaagtgaaaaaggccctaggactaagatcatggataagaggtcagcgtgcaatggccttagct

SEQ ID NO: 6 - DNA, 401 bp, Canis familiaris
  misc_feature (201)..(201): n is g or t
tcttgtgaaaagtagtacaggttttatttctattcatgtctattttatatattttttacaaaaatgtggaaaatcccaaggtacaatttttcatccaattggcaatttatccagtctctgaatggcttccaggtcctttccatcttgggcagaatccaagcagaacctaaaggaaattccctccctccttttttgcgtgcntatcctgtcaatattgctaaaaacaatattttagcaagtcaagataaatgcatacggtttaaatgttaaaactatgttggaaatagattgcttgagagaaaacaagactgcaaatagaataagaaaagaaaaaggaatataacaaatcagaaaagaacaagaaaaatcaaagattataaattagttctggctttaaaaaa

SEQ ID NO: 7 - DNA, 401 bp, Canis familiaris
  misc_feature (201)..(201): n is a or t
gagaaatacaaatgttgaaaagttgacatcatctgttaaagctaatctatatcaagtgttcttttaactgagctgtgattaataccatttgcatagaattagtctcctcggctacaattctgttatattttgcgttttttttttttttaaatatgacaatgtttgctataagaaagtaagaaggaaaaaaaaatccttccnctccttttggccagaaaagtgtaatagaaaataaattcccattttcaatttgaatgtgtacttaagtctttattgtatttagaagctgaagttctttgaattttaaaagcttgatgaagaagacttagattactaatctagttcaaaatgaaatcgattagataccaatttttattttttttatttttttttaatttttt

SEQ ID NO: 8 - DNA, 401 bp, Canis familiaris
  misc_feature (201)..(201): n is a or c
gactgatggaagatggcttgcttacccaagctactctgggtcttcctctcttaaccacaacctctgcaacacctgtagcgccccttgtctgtgtagctattcaagcaaggtatcagcctgcttagcagatcaagcaagtaatatccatactgaagtcctatgctgctaatgtattgactttctcactccacactactcacnaagcagactagacaacaaaaggtaagtcacctctacacctggtatgtgcctgaaggacaacaacaattttaattcctgcatcaagcaatcctcagaacaataccctgataggctgaaaaaaatcagattagtcatccacctctcgagtttcattgtaaagagtttggtgaaagaagtaaatattcttgtaagaatttgtc

SEQ ID NO: 9 - DNA, 401 bp, Canis familiaris
  misc_feature (201)..(201): n is c or a
tcttaaatacaatagctagttttgacttctatcgagctgatttagcactaatcattcaccaaattttgcagtattattttttaaaattactttcatcttcttaagggtttagctacctctcaattcctcatgtcccttccacctctcacctctcctccactctcaaagtggaaagcaaacatttgttggcatattccgtcnatgtgctcattttttaaaaaagatttatttagaaggagagaaagagagagcaggggggagggaaggaggaagagggagaggatctcaagcagactccatactgagtggagcccaatgctaggctggatcctaggatcctgaaatcatgacctgagttgaaaccaagagtaggacgtgacatttaaccaactaaaccaccc

SEQ ID NO: 10 - DNA, 401 bp, Canis familiaris
  misc_feature (201)..(201): n is a or c
cgtggaggctgcttctccctctgcctgtgtctctgcctctctctctctctgtctcttgtgaataaataaataaaatctttaaaaaaaaaagaaagaaattctctgcttgttggaaaaagattcattaagaagtgaccaatgctctgatacagttatatccatttgtctctggggtttttcttagcattttaaaatgagctnatagtgaaaaccaattaacaaacaaatgaggttacattcaaaaactgcaaagccaaacacattcctagtaaatacaaatactaataattagggttttaattagaataaagtttgaaccaaagaaagtatggaaataaacatagcaagtcaatatcgtaacaatcaactattttggcaggaaatatatgagcatgcaagta

SEQ ID NO: 11 - DNA, 401 bp, Canis familiaris
  misc_feature (201)..(201): n is a or g
ggagagatattcatctgtttgatggggtggtggtgagggaatggaaatgacattgctgttttaattcagagaagcaaagggtatcccacataggtctcagcatttttactgcattaaacctcttatttccatgtaatgtgcacacccacctcaaatacagacttcatcctaaatagccccttatatgatatgacagttttnttgctaacacaagaaaactacaagccacagtttgattcgctaaatcataaaaggcacctatttatcacaaaaaagctatattcgagctaattttccattcggaaacagtaggtaaaatacaacaatttacatagacataggtttaaccattcttacaagtacttcttcattcacctttgccacacttatctacaattata

SEQ ID NO: 12 - DNA, 401 bp, Canis familiaris
  misc_feature (201)..(201): n is t or c
gtcagaacagggcctctcattcctggcatcagagctccttccacagcgcccgcacggtgagggaagccgggcccagtgtcaccgtggatgcgggcggcaggaggatgcacatgaccgttggccaagtggcagcatgtggaagtggcaacgtgctcatggagagaagcactttgaccgacgcccagctcgggtgagtcttcnctaggtctgcgggacagtccgtgccagcacattctgttcccacagccctttagtgtgtgagctggcacttctggggctgcaaagaaaggcttccagagataagtgattagaataatgacttttcaggcagctgcctaattgacagattagcctatttacctcagggacaggggccaagctaacctgttcctatggcagct

SEQ ID NO: 13 - DNA, 401 bp, Canis familiaris
  misc_feature (201)..(201): n is a or g
atgaccgttggccaagtggcagcatgtggaagtggcaacgtgctcatggagagaagcactttgaccgacgcccagctcgggtgagtcttccctaggtctgcgggacagtccgtgccagcacattctgttcccacagccctttagtgtgtgagctggcacttctggggctgcaaagaaaggcttccagagataagtgattanaataatgacttttcaggcagctgcctaattgacagattagcctatttacctcagggacaggggccaagctaacctgttcctatggcagctttgtagggaaatatgacatttagtccgtctgttccaaattagttgctggctgaagggatgaccaccccttggccatgtgcagatctgctctgagcgagagctaattccag

SEQ ID NO: 14 - DNA, 401 bp, Canis familiaris
  misc_feature (201)..(201): n is a or c
cccagtgctcatcccgtcaagtggggcttgcttttttatcttaaaaaaatatatacacacactaagtgttgatgaaacttttcaccagatctcataaaaccaaactgtcaaaattgtggacctgactatgtataaacttgcaataaattattttggaaacataccttgtgcctgggttgtaccttgcggaaaagcaaatanagtaaatagaagttgcctacactgaagagaagattgacaaatctgagcattccgatggagcagaccacgtgttcagaccatccaaagatccagctggcgggtttgaccactccaactgacagcagatataggccaggtagcgtcgtaatcatgggatcccacttaaaaaaaaggaaaggccaacaatgaagcaagagcaa

SEQ ID NO: 15 - DNA, 401 bp, Canis familiaris
  misc_feature (201)..(201): n is t or c
gctgaaggcagacacttaaccatctcaggcacccctaaatctttttttattcccaacttaaaaaaacttggcattattatgcatgctttcattttctaatctggaaatacttgaatttttcttttgttactcaagttgagctagatttctactatctcttaagttactaatttgattataaatatcattttgcttacatanaggattaatgttgtgcttgccttcttggctcttattttaaggctaggataaacactggttttgagaataatgaaacgtgaaaatcacactgaaacctaaaaatatatatgcatggtagtaaaaattaagaattataacccaagaacattaacacaatcctgctgacattaagcatggtgctatagtgtttaaatttatgt

SEQ ID NO: 16 - DNA, 401 bp, Canis familiaris
  misc_feature (201)..(201): n is a or g
gagagccttatggaaggctaagaaaaaagaagggaaaggaccagaaaaaatgaatatactacataggatgtccggaattgggaggatactgatggcagggggtgtgtttcaggggatcaggtgtctttttggaaactattctgatagtactgtggaggattttaggagggtgaaatagaaaccaggtcagttagcaaaatntaatcatattcaaggtgaaatgtcgttaattacatctgaactctggtgaagtaagtacaatggagggaaatagatatgttaaaacctaataagagtgaattgaaaggtattagtaatcatggatgaagtcatgtggaggaaggaagagttgactcctgggcttcagtattaaatgaaaggatattgccatttattaaaat

SEQ ID NO: 17 - DNA, 401 bp, Canis familiaris
  misc_feature (201)..(201): n is g or a
aactgcaggtaccctaacctgctcacacaccaaaatcctccatttctttgagtttcagaccaatcagttgaagagaccgtcccatgctgggcttttcttgatccttaggatactggatgacagagacaatgaatggcaagatttcagcctagatcctctcgacttttaagggttaattttgaaaattaaaatgatgtcaanttgttatcttaatagaattgtcaagtaaatcaatgatcaaattcagcctcccctccccccccacccagtacctgcaatggtcaattctgaagggaaattaagaaagacttcacagatattggtacagattaaatcttttgaaaacaaagtttccaaattaaaaaaaaaaaacatattctaactcatttaatataacaggt

SEQ ID NO: 18 - DNA, 401 bp, Canis familiaris
  misc_feature (201)..(201): n is a or c
acagtttaggaattaatttagcagtcttcaaagatacaaaggatatccaattgagaggcaaaggaggaaatcaaaccttcaatttctgctaagatgtaaggatagtataaactagttagaaaggttttttgcctaggaggcaattcaacaatgattgggcacatgaaaaaattgtattatcttctctttgaaataatatcntatttcaaaacaggattaagtgtggtcctatcaggatacaggttagtagaatagatgacctcctaggttcccttttcatcctaagatttttatcttttatgatagcagtcagagacgttctgctgaacaaaattgatttctggcaaaggacatatctccaaagaaccatttttcatgggtttgtatataatttttattga

SEQ ID NO: 19 - DNA, 401 bp, Canis familiaris
  misc_feature (201)..(201): n is t or a
gaaagatcattatttgagttagcacagacaatattctcagaataagaacttaaggctttaaatataatcagaaatgggatctaaaatttgaaggggaaaaaagaaacccaccctgtttccacaaagtagacaacaaagagtaaaacagaattgggaactcgcaaaagtatttttgatataagggttttaaaaggcatcaangaatctttaccaaccctgcagtactatgaagtttgtagtttatctacttatgactacgacaagtactcttttaaaaaatccaaatgtgtacctatatattgggacagggtacacttaaaagattagttcttgtaaaaagtttagtaatttcttaggttggagtttttttttttgttttttttttttttaattttttttat

SEQ ID NO: 20 - DNA, 401 bp, Canis familiaris
  misc_feature (201)..(201): n is g or a
cccaaagaactaaggaatgatgaaggtcagagcatcactatctatccatttagataaatcagttatgagtagggtggaaggagagggaccgaactactctgggaaaaagcaactgttgagaacaaaagggcattaatttcttctcaagtaaatcattttaaaattcctggtgaaaaaattatagtgacagtataggtcagntgtcttcagtagattcagtcagtggtaaccaagataggtcctgtacagggtaaactgggcagccagaaactttccccattggacttgggggcaggaccacagagaaaagcaaaagttggagcttggcagataccttaaaaataatcatttaatacgtgcttccagtagctacaaaggagtgaagcccttcttaggtagct

SEQ ID NO: 21 - DNA, 401 bp, Canis familiaris
  misc_feature (201)..(201): n is g or a
aaaactcaattataaggctatttaataggtgaactaaaaaaagacatctagtcccctataacaataacctcagtttcaaagaaatcacttgtgggaggtcaacttcaaaagcaaaatccaaatctaatattctgatgcttggttatttctatacaatctgtacaaaggccaaggatgttgttaaacactctactaacaggnaagatatcccctcgcactcacaaaaaaagaagtagctggattaaaaaaaaaaaaaaaaaaaaaaagccgatagtgcccaaggttgtgagatacttcctttaccaaagcttcactcagtgtaagattttttaattcaactgagtcttttgtatttcaacgtatttctacttagttattccactaatttatcctcaaaaaat

SEQ ID NO: 22 - DNA, 401 bp, Canis familiaris
  misc_feature (201)..(201): n is a or c
agatggtcagcctcatgacttaggaaaagttggagaggtcattgtgaccaaagatgatgccatgcttttaaaaggaaaaggtgacaaggctcaaattgaaaaacgttttcaagaaatcattgagcagttagatatcacaactagtgaatatgaaaaggaaaagctgaatgagcatctggcaaaactctcagttggagtagntgtgttgaaggttggtgggacaagcgatgttgaagtgaatgaaaagaaagagttacagatgccctcaatgccacaagagatgctgttgaagaaggcattgttctgggagggggttgtgccctgctttggttcattccagccttggattcaataactccagctagtgaagatcaaagaattggtatcaaaattattaagag

SEQ ID NO: 23 - DNA, 401 bp, Canis familiaris
  misc_feature (201)..(201): n is t or c
tttctgtatggccagcagtatcaaagggctatcatgaaataactgtaaacaatttcattttccattattcaggcttatggagcaagttttaaactcttctcagttcttaaacacttccttggatttattatatatgtttataacacatatatgtgatgcttaaaacacaaaaaaatctgatgtgacctgtggatcaccacngtgcctaaaagaaagtccataatttttacctgttcagttgttcattgtggagatagtctaaataagaagtctatttttaggattcaagatatctctgatgttaccacataaacctctggatgtcctgactatttggccactgaaaagtcttgttcaagaagatataaaaagttaggaaattgacaactgcatagcaaccc

SEQ ID NO: 24 - DNA, 401 bp, Canis familiaris
  misc_feature (201)..(201): n is a or t
tgatactattggctaattatggccctaaggaagaaatttttctaaaatgggcttacatttttaagggagaaataacttgttttcctgcccttgacttggcaccatcaatacaattctatttttctctttttttcagaaaggattctttgatcctggacattggtattttgtcctccactcttaccaggcacttaacaattntatttataaacttgacaagagattttagaaacaggctgagagcctatgatggggatatttacttcattcttcttgcttctgttttcttttcataagaagacactcgttccttataaacatcttccttaactgtggtatagccctagggagtctgtgtagtcactgtgacctagctagcctgccctcgtgtcaggcaatat

SEQ ID NO: 25 - DNA, 401 bp, Canis familiaris
  misc_feature (201)..(201): n is c or t
ggagacaaagaaattgacatcatttctgatgcaatgaaaaaggttggaagaaagggtgtcatcacagtaaaggatggaaaaacactgaatgatgaactagaaattattgaaggcatgaagtttgattgaggttatatttctccatactttattaatacatcaaaaagtcagaaatgtgaattccaagatcctatgttctantgagtgaaaagaagatttctagcatccagtccattgtatctgctcttgaaattgccaatgctcaccgtaagtccttggttataattgctgaagatgttgatggcgaagctctgagtacacttgttttaaataggctaaatgtcagtcttcaggttgtggcagtcaaagctccaggttttggtgacaatagaaagaaccag

SEQ ID NO: 26 - DNA, 401 bp, Canis familiaris
  misc_feature (201)..(201): n is t or c
cttgactcttaaaatgtcagacatcctgcaagatgaagagcagcatgcaatgggctggagaaagagaaacagcagaaagtagagttggaagggcccagggaacgatccatcacaaagccatcctgcctcaacctgaaaagtgcaattccctgagattccagcaacactcatgactgaacctcctgcttttcttagcaaaanaataaatacgcatgagccttgtttattcaattgactctagccgaatgagaaggggaagctccagactgagaagaaataaaaaccaggagctctcctttttttttttttttttttttgagtgaaaccattattaataaacatttgtgaagacccagttgctctcaaaagatcagaactggtatgattcactgtaggcagct

SEQ ID NO: 27 - DNA, 401 bp, Canis familiaris
  misc_feature (201)..(201): n is a or g
gatattcatttctcatatgtactttttgagatgtttgtctgtgagtatgtacatatcttttaatggtgtacagataaggaaaagggagaaagacagtgagattatataaggaagaaaaagatcagatgcatgggaaccactagtcttctacaatgaaaaagtaagtcaggacttaaattcagctaatgccaatcatgtacngtttcatgctttgtagtgttttattaaacaaaatcactggatgaagaaaagtttagctaaatctattattttaaatgctaatgattttgattttcagtgttcaaaggggtccctctaatcagccgagaaaaattgttctgtccctgtaaatagtaggtagaatgcttattgcaattcctcttattatataaagatatgct

SEQ ID NO: 28 - DNA, 401 bp, Canis familiaris
  misc_feature (201)..(201): n is t or g
tacgagagaaggtctaaaatgatcagacctcaacatggagctgatttaagctcctggtaaattgctcaaatctggatatagaagactttggtttactccaatctagcaagaatgtagctttttaaaattgctactctgacctaggttattactcccatcaaagtttcttcacagcttctaaattaagaaatcattttgaantagaaggaaaaaaaggaaatataaggccattaaattatgttctctggaacaatggtaaaacaaaaataaaaacgtaatagtcacagataaaaaaatgttttaataaatgaattacattgacccagctgttaaaacctgattttggacttgcacaacttttaacccagagagggagagaatgaaataacataactttattg

SEQ ID NO: 29 - DNA, 401 bp, Canis familiaris
  misc_feature (201)..(201): n is a or c
ttgttgctaatatttatttgaatatgaagctgtttcaaaaattgtcttttgccttgtttgaagcttgttttataagatagagaaaaacttagtttttcctaggagcattactaaaaatctttaacaatacatgattataaatttattttcttgttgtcacatttatttagcaacttaaactgtatttataattaggtaatnattttcccttgaaatacctgttttttggcaaatggtttcatttaaactattttaatgaggctatttaataaaaatatttttcttataatactatttataatatatatgcagttatatttatgcagcaataaataaataacttgccaaggtgagtcacttaagagcaaatgtttcaagtgttaaagtatttaattaaaaaa

SEQ ID NO: 30 - DNA, 401 bp, Canis familiaris
  misc_feature (201)..(201): n is a or t
attagcacttggcattgtatctcatggagtcattttaaatggtgacacttactacaagaaagtgcaatcttcatttatctgtgaaatgccagataaatacctttgaagatatttgataaacacacttggtgttttatttctgcaattatgcatcatcatggtgtgaaataacagtgattcttgatccagtgcttcactcangaacttacttgtaagtagtctaaaacatttagaactcatttaggaggagcatgtataaacctttaatatttgtcttatttattttaaatttcctcacttattctaattaatctttccctaaaagaaagatttagccctcttcttgcttgcagtaacctgaaggtcctaaatgtgtattcaatagacaagattcagcctgt

SEQ ID NO: 31 - DNA, 401 bp, Canis familiaris
  misc_feature (201)..(201): n is c or t
aagattagcaaaggtgctaatcctgtggaaatcaggagaggtgtgatgttagctgttgatgctgtaatcgctgaacttaagaagcagtctaaacccatgacaacccctgaagaaattgctcaggttgctacaatttctgcaaatggagacaaagaaattgacatcatttctgatgcaatgaaaaaggttggaagaaagggngtcatcacagtaaaggatggaaaaacactgaatgatgaactagaaattattgaaggcatgaagtttgattgaggttatatttctccatactttattaatacatcaaaaagtcagaaatgtgaattccaagatcctatgttctattgagtgaaaagaagatttctagcatccagtccattgtatctgctcttgaaattgcc

SEQ ID NO: 32 - DNA, 401 bp, Canis familiaris
  misc_feature (201)..(201): n is c or g
tgaacagttttagagtaaccatctttaaacctcataaggcagagttgttgcttatttaatatttcaacaaaccacccctggccccatcatctcccattgttcttactgcctggaccttggagatgctcttaaggtgcatctaagagaaagatgaccactgagctcttaccatggaaagagcaaaagcaaactgttctcaanatttgctcctttagttttaaagcagcaatggccatgaccttgagattttcacatcataaacttattcattcatttaatttccatgaaacaggactgctggacattcctaatatatttagataagctgatttcatacagataggctttcctattagtcaaagaggtggatatcattgaccttacaaattaggaggaaggat

SEQ ID NO: 33 - DNA, 401 bp, Canis familiaris
  misc_feature (201)..(201): n is a or g
aatacaattctatttttctctttttttcagaaaggattctttgatcctggacattggtattttgtcctccactcttaccaggcacttaacaattatatttataaacttgacaagagattttagaaacaggctgagagcctatgatggggatatttacttcattcttcttgcttctgttttcttttcataagaagacactcnttccttataaacatcttccttaactgtggtatagccctagggagtctgtgtagtcactgtgacctagctagcctgccctcgtgtcaggcaatattcagaccagccagggcctcactgtcacctccattcactgtggcttgccatgcctagaaaacaaagggcaacagtaattacttctcaaaaggtaggcacctgggaag

SEQ ID NO: 34 - DNA, 401 bp, Canis familiaris
  misc_feature (201)..(201): n is g or a
ctgcctaagataagccttggtaattctctgattcccacaaacttaagtattggctgttgagaaaaatattttagtactttaagataaatatatcactttaataattactgcttattttcagagtggtacaataacaaattcctttctgactagtgacttctattcatggatatagcactataaaataatccacccttatcnttcatatttctgggtagttgagtaagataggaactatttttgatatgcaggagcaaatactgaagcagtggggttacactttctgtcaacaaaggtatcaactgccagaaaaccctgataagtcccatttcaagcaagtagagtctggataataccaaactacaagacaagagtcattgcgtgtgaactggcggtactgc

SEQ ID NO: 35 - DNA, 401 bp, Canis familiaris
  misc_feature (201)..(201): n is t or c
atgagcctatgccatcacttttggaaactacttatagctgagcaacacatgggatgtactactctattctttttttgccctgagtgtccatttgctctgtcttaaaaaaaaaaaaaaaaaaatcaacaaagagcttttaccagttatgcccactgccctttccttgttagtgttgtatagctgatgtgtaatttaggggcnagcctggaaatcagttgccatttatagcattttttctttgaataaatgatttccaagtgccacacaaccaaccttttttttttcacacaaccaacttataaacaaactatgatataaaacccttgcaattcagaaatatttttcaattttaaatattaaacacccattgctttcatggatttattagtgcctcagtatta

SEQ ID NO: 36 - DNA, 401 bp, Canis familiaris
  misc_feature (201)..(201): n is a or g
gagtatttcagcaatgaaaccaatgcaaatatgttgtacaaaatatgtagtgtaaagacagtttgagaacttatgagctttaatcctcttatctattttaggactttctgggacaagtgttttgtacattgggggagattgttggttctcagggaagtcgcctggaaaagccaatagtgtaagtattttttaatccagacnatgaaatgttaactcttcagttttcattttggtagtttggagttgatgaatacatggttgtagaccaaagtggttttaaaaatcttattgaagtttttttctaacaaagagttttgacatgtttgtaaagaatcatttctatttcagctgacatatgccatgctgtaaaatgttcgcaaaatgaaatgagattttaaaaa

SEQ ID NO: 37 - DNA, 401 bp, Canis familiaris
  misc_feature (201)..(201): n is g or a
tcattctgtcatccaggcatttctatttcccagtgagacctaagcctcaatctcgtcttaaatgtatgcagtatctattaccaatgccatttgatatggcacaaccttttcacaaagtacctgctggggagtaatttcttcttgcataatttctacattgatttccaagacaccaatggcctgaataaaaaccaaggtgcntattaatctcctgcttacttgaattcttttattcaggaattattttcatgtattaaccttagtgtgaacaattttcaacacctaacctacagaaagaaattagagacagagctcataggcaggtttgagctgacaggaacgtcatttaaggtcacaatggggtcaatggtccacttgtttcagccttttcatgtgagctc

SEQ ID NO: 38 - DNA, 401 bp, Canis familiaris
  misc_feature (201)..(201): n is c or t
ctgaatgtttaagctgaccagagaaaagttatacaggaatagctgtttaaaggaggactggaaatttggaattctcaagcaagacagttgcaaaaacatcccaagagttgtttgtgtagagaaccaaaggagacagtgaaaataaaagaagcagtcttaatctcctaaacaaccaaactttttaggggccttttgaaagcngctaaacatcactcctacagaacaaattcttctgctaccatcccttcctctgcatttggctaagaaaacatatgctctatagccctggaatcaccttatgacccccataactcctgcactgcagctgaggactcttagtttaccaactcatctccacttttctttccctcccaggccattccagagacttacaaataaag

SEQ ID NO: 39 - DNA, 401 bp, Canis familiaris
  misc_feature (201)..(201): n is a or c
ttcttcttttttttttttttttttttttttttttttaagaaaaggggccaacttcttcatgtgattcagagaaatgggcaccttatgtaataggcctggagtccttggtctttaacaaagatgaaaactgatttatgcctcatttcaaccttctttctctgcagacaggcaccactgccacttatgaaagccgctgggggngaggaactgcacagtacagctcgcagaagtcagtggaagaaagatccttgaggcagcctctgaggagactcgagatttctccagacagcagccctgagaggattcattactcacacggtgattaccagcacggccagagaagccaggccgggcacactctgcggcaccaagacagcaggaggtctacccttcttctgcca

SEQ ID NO: 40 - DNA, 401 bp, Canis familiaris
  misc_feature (201)..(201): n is g or a
taacagcagtggcagaagcagcagccgtgttattggcattaaggatctaccaatcaagttgaccaatcctatgttttcataaaatttgtgaagacatagattgcatctgccaacactagttagcttactttttttaatagtttattagagacattattaatgtcaacattactcatacactagaaaattcatcatgtgcantttttagaaaatcaaatttttgtcctcttttaccactgataccccccccatagacatgtcacccactaaattcatcttcatgcataatatgatttaatactagaatgcttagtggaaatacaaaacaatatatctgcctgaaatgttatttttcagagagactgtggctaatatacataatagtttttgaactgcattgg